(19) United States
(12) Patent Application Publication
(10) Pub. No.: US 2018/0111988 A1
(43) Pub. Date: Apr. 26, 2018

(54) PHARMACEUTICAL COMPOSITION FOR TREATING RHEUMATOID ARTHRITIS

(71) Applicant: Eisai R&D Management Co., Ltd., Tokyo (JP)

(72) Inventors: Nobuyuki Yasuda, Tsukuba (JP); Fumitoshi Tago, Tokyo (JP); Hiroko Baba, Tokyo (JP); Tomohisa Nakano, Tokyo (JP); Masahiko Mori, Tokyo (JP)

(21) Appl. No.: 15/791,674

(22) Filed: Oct. 24, 2017

(30) Foreign Application Priority Data
Oct. 26, 2016 (JP) 2016-209630

(51) Int. Cl. C07K 16/24; A61P 19/02

(52) U.S. Cl. CPC C07K 16/24; A61P 19/02; A61K 2039/505; C07K 2317/52; C07K 2317/24

(57) ABSTRACT

An object of the present invention is to provide a pharmaceutical composition comprising an anti-fractalkine antibody, and a method for treating rheumatoid arthritis using the pharmaceutical composition, wherein the pharmaceutical composition and the method bring about therapeutically effective amelioration in rheumatoid arthritis after administration to a human subject. The present invention provides a pharmaceutical composition for the treatment of rheumatoid arthritis. The pharmaceutical composition in a method for treating rheumatoid arthritis comprises an anti-fractalkine antibody and a pharmaceutically acceptable excipient and is used such that at least 100 mg per dose of the anti-fractalkine antibody is subcutaneously administered to a human in need thereof.

### BACKGROUND OF THE INVENTION

### Field of the Invention

A portion of the contents related to the present application was published within 1 year before the effective filing date of the present application (Oct. 26, 2016) by the inventor related to the present application or a joint inventor(s) thereof, or by a third party who obtained the information of the invented object directly from the inventor related to the present application at the request of the applicant related to the present application, in a mobile app of the meeting proceedings of American College of Rheumatology (publication date: Oct. 27, 2015), the following URL (http://acrabstracts.org/abstract/safety-and-efficacy-of-e6011-an-anti-fractalkine-monoclonal-antibody-in-a-first-in-patient-phase-12-study-in-rheumatoid-arthritis/) (publication date: Oct. 27, 2015), American College of Rheumatology (publication date: Nov. 10, 2015), the conference program/conference abstract of The 60th Annual assembly and Scientific Meeting of the Japan College of Rheumatology (publication date: Mar. 18, 2016), a mobile app of the conference program/conference abstract of The 60th Annual assembly and Scientific Meeting of the Japan College of Rheumatology (publication date: Apr. 8, 2016), The 60th Annual assembly and Scientific Meeting of the Japan College of Rheumatology (publication date: Apr. 23, 2016), a mobile app of the meeting proceedings of European League Against Rheumatism (publication date: Jun. 8, 2016), and the following URL (http://www.abstracts2view.com/eular/view.php?nu=EULAR16L_FRI0236), European League Against Rheumatism (publication date: Jun. 10, 2016), respectively.

Also, a portion of the contents related to the present application was published within 1 year before the filing date of the present application by the inventor related to the present application or a joint inventor(s) thereof, or by a third party who obtained the information of the invented object directly from the inventor related to the present application at the request of the applicant related to the present application, in the conference program/conference abstract of The 61st Annual assembly and Scientific Meeting of the Japan College of Rheumatology (publication date: Mar. 18, 2017), a mobile app of the conference program/conference abstract of The 61st Annual assembly and Scientific Meeting of the Japan College of Rheumatology (publication date: Apr. 5, 2017 or later), The 61st Annual assembly and Scientific Meeting of the Japan College of Rheumatology (publication date: Apr. 20, 2017), a mobile app of the meeting proceedings of European League Against Rheumatism (publication date: May 14, 2017), the following URL (http://scientific.sparx-ip.net/archiveeular/index.cfm?searchfor=E6011&view=l&c=a&item=2017SAT0187) (publication date: May 14, 2017 or later), European League Against Rheumatism (publication date: Jun. 17, 2017), Modern Rheumatology (2017) (http://dx.doi.org/10.1080/14397595.2017.1337056, publication date: Jul. 6, 2017), a mobile app of the meeting proceedings of American College of Rheumatology (publication date: Sep. 22, 2017), and the following URL (http://acrabstracts.org/abstract/safety-pharmacokinetics-and-efficacy-of-e6011-an-anti-fractalkine-monoclonal-antibody-in-a-first-in-patient-phase-12-study-on-rheumatoid-arthritis-52-week-results/) (publication date: Sep. 18, 2017).

The present invention relates to a pharmaceutical composition for the treatment of rheumatoid arthritis, comprising an anti-fractalkine antibody, and a method for treating rheumatoid arthritis using the pharmaceutical composition.

### Description of the Related Art

Fractalkine (also referred to as “FKN”) is a membrane-bound chemokine that is expressed on the surface of vascular endothelial cells by the inflammatory stimulus of LPS, TNF-α, IL-1, or the like. Cells expressing an FKN receptor CX3CR1 bind to membrane-bound FKN without any involvement of selectin or integrin and cause strong cell adhesion. Secreted FKN shed from the membrane-bound FKN exhibits cell-migrating activity against NK cells, T cells, and monocytes having CX3CR1.

The expression of FKN is induced on the surface of vascular endothelial cells by proinflammatory cytokines. The increased expression of FKN and the accumulation of CX3CR1⁺ cytotoxic effector lymphocytes and macrophages have been reported as to patients having rheumatoid arthritis (also referred to as “RA”).

Therapeutic effects by the inhibition of FKN have previously been reported as to collagen-induced arthritis (CIA) murine models, which are known as chronic rheumatoid arthritis models (J Immunol. 2004; 173: 7010-7016). The results obtained in CIA indicate significant reduction in the clinical score of arthritis, significant decrease in the incidence of arthritis, and significant decrease in inflammatory cells in synovial membranes and bony erosion, due to anti-fractalkine antibodies. Furthermore, it is suggested that an anti-fractalkine antibody inhibiting the interaction between FKN and CX3CR1 is capable of treating inflammatory diseases including rheumatoid arthritis (WO2006/046739). Thus, as for the treatment of rheumatoid arthritis, it is suggested that an antibody that can bind to FKN and inhibit this FKN is effective for the treatment of rheumatoid arthritis.

The applicant of this application has previously disclosed a plurality of mouse anti-human fractalkine (hFKN) monoclonal antibodies (clones 1F3-1, 3A5-2, 1F3, 1G1, 2B2, 3D5, 3H7, 6D1, 7F6, and 5H7-6). Particularly, the clone 3A5-2 has been humanized because of its high neutralizing activity, binding affinity, and interspecific cross reactivity against hFKN, and designated as H3-2L4 (WO2011/052799, which is incorporated herein by reference in its entirety).

An object of the present invention is to provide a pharmaceutical composition comprising an anti-fractalkine antibody (in the present specification, also referred to as an “anti-FKN antibody”), wherein the pharmaceutical composition brings about therapeutically effective amelioration in rheumatoid arthritis after administration to a human subject.

Another object of the present invention is to provide a pharmaceutical composition comprising an anti-FKN antibody, wherein the pharmaceutical composition is used in such a manner that therapeutically effective amelioration in rheumatoid arthritis is achieved.

A further alternative object of the present invention is to provide a method for treating rheumatoid arthritis which brings about therapeutically effective amelioration in rheumatoid arthritis.

A further alternative object of the present invention is to provide a method for treating rheumatoid arthritis using a pharmaceutical composition that brings about therapeutically effective amelioration in rheumatoid arthritis.

### SUMMARY OF THE INVENTION

The present invention encompasses the following embodiments:

[1] A pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the pharmaceutical composition is used such that at least 100 mg per dose of the anti-fractalkine antibody is subcutaneously administered to a human.

[2] The pharmaceutical composition according to [1], wherein

the pharmaceutical composition is used such that 100 mg to 400 mg per dose of the anti-fractalkine antibody is subcutaneously administered to a human.

[3] The pharmaceutical composition according to [1], wherein

the pharmaceutical composition is used such that 100 mg, 200 mg, or 400 mg per dose of the anti-fractalkine antibody is subcutaneously administered to a human.

[4] The pharmaceutical composition according to [1], wherein

when 100 mg of the anti-fractalkine antibody is subcutaneously administered by a single administration to a human,

mean Cₘₐₓ is a value that falls within the numerical range of 80% to 125% of 10 μg/mL,

mean AUC_{(0-336 h)} is a value that falls within the numerical range of 80% to 125% of 2.4×10³ μg·h/mL, or

mean AUC₍₀₋ₜ₎ is a value that falls within the numerical range of 80% to 125% of 3.0×10³ μg·h/mL.

[5] The pharmaceutical composition according to [1], wherein

when 200 mg of the anti-fractalkine antibody is subcutaneously administered by a single administration to a human,

mean Cₘₐₓ is a value that falls within the numerical range of 80% to 125% of 27 μg/mL,

mean AUC_{(0-336 h)} is a value that falls within the numerical range of 80% to 125% of 7.4×10³ μg·h/mL, or

mean AUC₍₀₋ₜ₎ is a value that falls within the numerical range of 80% to 125% of 1.2×10⁴ μg·h/mL.

[6] The pharmaceutical composition according to [1], wherein

when 400 mg of the anti-fractalkine antibody is subcutaneously administered by a single administration to a human,

mean Cₘₐₓ is a value that falls within the numerical range of 80% to 125% of 43 μg/mL,

mean AUC_{(0-336 h)} is a value that falls within the numerical range of 80% to 125% of 1.2×10⁴ μg·h/mL, or

mean AUC₍₀₋ₜ₎ is a value that falls within the numerical range of 80% to 125% of 2.7×10⁴ μg·h/mL.

[7] The pharmaceutical composition according to [1], wherein

the pharmaceutical composition is subcutaneously administered by multiple administrations at dosing intervals of once a week to once every two weeks.

[8] The pharmaceutical composition according to any one of [1] to [7], wherein

the pharmaceutical composition is subcutaneously administered such that the mean trough concentration of the anti-fractalkine antibody is 10 μg/mL or higher.

[9] The pharmaceutical composition according to any one of [1] to [7], wherein

the pharmaceutical composition is subcutaneously administered such that the mean trough concentration of the anti-fractalkine antibody is 20 μg/mL or higher.

[10] A therapeutic agent for rheumatoid arthritis, comprising an anti-fractalkine antibody, wherein

the therapeutic agent for rheumatoid arthritis is used such that 100 mg to 400 mg of the anti-fractalkine antibody is subcutaneously administered at dosing intervals of once a week to once every two weeks to a human, and

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In another aspect, the present invention relates to the following inventions:

[1′] A method for treating rheumatoid arthritis,

the method comprising subcutaneously administering at least 100 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

[2′] The method according to [1′], wherein

the method comprises subcutaneously administering 100 mg to 400 mg per dose of the anti-fractalkine antibody to a human.

[3′] The method according to [1′], wherein

the method comprises subcutaneously administering 100 mg, 200 mg, or 400 mg per dose of the anti-fractalkine antibody to a human.

[4′] The method according to [1′], wherein

the anti-fractalkine antibody is subcutaneously administered by a pharmaceutical composition which brings about the following:

when 100 mg of the anti-fractalkine antibody is subcutaneously administered by a single administration to a human,

mean Cₘₐₓ is a value that falls within the numerical range of 80% to 125% of 10 μg/mL,

mean AUC_{(0-336 h)} is a value that falls within the numerical range of 80% to 125% of 2.4×10³ μg·h/mL, or

mean AUC₍₀₋ₜ₎ is a value that falls within the numerical range of 80% to 125% of 3.0×10³ μg·h/mL.

[5′] The method according to [1′], wherein

the anti-fractalkine antibody is subcutaneously administered by a pharmaceutical composition which brings about the following:

when 200 mg of the anti-fractalkine antibody is subcutaneously administered by a single administration to a human,

mean Cₘₐₓ is a value that falls within the numerical range of 80% to 125% of 27 μg/mL,

mean AUC_{(0-336 h)} is a value that falls within the numerical range of 80% to 125% of 7.4×10³ μg·h/mL, or

mean AUC₍₀₋ₜ₎ is a value that falls within the numerical range of 80% to 125% of 1.2×10⁴ μg·h/mL.

[6′] The method according to [1′], wherein

the anti-fractalkine antibody is subcutaneously administered by a pharmaceutical composition which brings about the following:

when 400 mg of the anti-fractalkine antibody is subcutaneously administered by a single administration to a human,

mean Cₘₐₓ is a value that falls within the numerical range of 80% to 125% of 43 μg/mL,

mean AUC_{(0-336 h)} is a value that falls within the numerical range of 80% to 125% of 1.2×10⁴ μg·h/mL, or

mean AUC₍₀₋ₜ₎ is a value that falls within the numerical range of 80% to 125% of 2.7×10⁴ μg·h/mL.

[7′] The method according to [1′], wherein

the method comprises subcutaneously administering the anti-fractalkine antibody by multiple administrations at dosing intervals of once a week to once every two weeks.

[8′] The method according to any one of [1′] to [7′], wherein

the anti-fractalkine antibody is subcutaneously administered such that the mean trough concentration thereof is 10 μg/mL or higher.

[9′] The method according to any one of [1′] to [7′], wherein

the anti-fractalkine antibody is subcutaneously administered such that the mean trough concentration thereof is 20 μg/mL or higher.

The present invention provides a pharmaceutical composition comprising an anti-FKN antibody, wherein the pharmaceutical composition brings about therapeutically effective amelioration in rheumatoid arthritis after administration to a human subject. The pharmaceutical composition of the present invention can be used in such a manner that a pharmacokinetic parameter useful for exerting therapeutic effects on rheumatoid arthritis is achieved.

The present invention also provides a method for treating rheumatoid arthritis, wherein the method brings about therapeutically effective amelioration in rheumatoid arthritis.

### BRIEF DESCRIPTION OF THE DRAWINGS

FIG. 1 shows, in a time-dependent manner, the summary statistic of serum H3-2L4 concentration after a single subcutaneous administration (each dose of 50 mg, 100 mg, 200 mg, and 400 mg) of antibody H3-2L4;

FIG. 2 shows time-dependent change in the summary statistics of serum H3-2L4 concentration by multiple subcutaneous administrations (100 mg group, 200 mg group and 400 mg group) of antibody H3-2L4;

FIG. 3 shows time-dependent change in the summary statistics of serum H3-2L4 concentration and serum total FKN concentration by multiple subcutaneous administrations (100 mg group) of antibody H3-2L4;

FIG. 4 shows time-dependent change in the summary statistics of serum H3-2L4 concentration and serum total FKN concentration by multiple subcutaneous administrations (200 mg group) of antibody H3-2L4;

FIG. 5 shows time-dependent change in the summary statistics of serum H3-2L4 concentration and serum total FKN concentration by multiple subcutaneous administrations (400 mg group) of antibody H3-2L4;

FIG. 6 shows ACR20, ACR50, and ACR70 (LOCF) at week 12 of multiple subcutaneous administrations (100 mg group, 200 mg group, and 400 mg group) of antibody H3-2L4;

FIG. 7 shows ACR20, ACR50, and ACR70 (NRI) at week 12 of multiple subcutaneous administrations (100 mg group, 200 mg group, and 400 mg group) of antibody H3-2L4;

FIG. 8 shows change in ACR70 over 12 weeks of multiple subcutaneous administrations (100 mg group, 200 mg group, and 400 mg group) of antibody H3-2L4;

FIG. 9 shows change in ACR20 over 52 weeks of multiple subcutaneous administrations (100 mg group, 200 mg group, and 400 mg group) of antibody H3-2L4;

FIG. 10 shows change in DAS28-CRP over 12 weeks of multiple subcutaneous administrations (100 mg group, 200 mg group, and 400 mg group) of antibody H3-2L4;

FIG. 11 shows change in the proportions of disease activity scores of DAS28-CRP in multiple subcutaneous administrations (100 mg group, 200 mg group, and 400 mg) of antibody H3-2L4 before administration and at week 12 after the start of administration;

FIG. 12 shows the proportion of subjects who reached remission or low disease activity in disease activity scores of DAS28-ESR in multiple subcutaneous administrations (100 mg group, 200 mg group, and 400 mg group) of antibody H3-2L4 at 12 weeks after the start of administration; and

FIG. 13 shows the proportion of subjects based on EULAR response criteria of DAS28-ESR in multiple subcutaneous administrations (100 mg group, 200 mg group, and 400 mg group) of antibody H3-2L4 at 12 weeks after the start of administration.

### DETAILED DESCRIPTION OF THE PREFERRED EMBODIMENTS

### 1. Summary of Invention and Definition

The present invention relates to a pharmaceutical composition and a method for treating rheumatoid arthritis, which bring about therapeutically effective amelioration in rheumatoid arthritis.

In one embodiment of the present invention, the “therapeutically effective amelioration in rheumatoid arthritis” can mean that the therapeutically effective amelioration is exhibited for one or more of items or parameters specified in evaluation criteria for rheumatoid arthritis established in the art. Examples of such items or parameters can include, but are not limited to, ACR (American College of Rheumatology) 20 response rates, ACR50 response rates, ACR70 response rates, erythrocyte sedimentation rates (ESR), high sensitive C-reactive protein (hs-CRP), health assessment questionnaire (HAQ), simple disease activity index (SDAI), disease activity score 28 joints (DAS28; e.g., DAS28-CRP using CRP and DAS28-ESR using ESR), clinical disease activity index (CDAI), and Boolean remission rates.

In one embodiment of the present invention, the therapeutically effective amelioration in rheumatoid arthritis can mean at least 10%, at least 20%, at least 30%, at least 40%, at least 50%, at least 60%, at least 70%, at least 80%, or at least 90% decrease in ESR from the baseline.

In one embodiment of the present invention, the therapeutically effective amelioration in rheumatoid arthritis can mean at least 10%, at least 20%, at least 30%, at least 40%, at least 50%, at least 60%, at least 70%, at least 80%, or at least 90% decrease in hs-CRP from the baseline.

In one embodiment of the present invention, the therapeutically effective amelioration in rheumatoid arthritis can mean that the DAS28 score itself or severity evaluated on the basis of DAS28 is ameliorated. For example, the therapeutically effective amelioration in rheumatoid arthritis can mean that, in severity evaluation based on DAS28-CRP or DAS28-ESR, a severe case is ameliorated to a moderate case, a moderate case is ameliorated to a mild case, or a mild case is ameliorated to remission.

In one aspect, the present invention relates to a pharmaceutical composition for the treatment of rheumatoid arthritis which brings about a therapeutically effective serum anti-FKN antibody concentration after a single administration or multiple administrations to a human subject. In another aspect, the present invention relates to a method for treating rheumatoid arthritis, comprising administering an anti-FKN antibody to a human subject in such a manner that a therapeutically effective serum anti-FKN antibody concentration is achieved.

In one embodiment of the present invention, the “therapeutically effective serum anti-FKN antibody concentration” is a serum concentration of 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher of the anti-FKN antibody. In an alternative embodiment of the present invention, the “therapeutically effective serum anti-FKN antibody concentration” can be a mean trough concentration of 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher of the anti-FKN antibody. In a preferred embodiment, the “therapeutically effective serum anti-FKN antibody concentration” is a mean trough concentration of 10 μg/mL or higher or 20 μg/mL or higher of the anti-FKN antibody.

In a preferred embodiment of the present invention, the pharmaceutical composition of the present invention is formulated in such a manner that a serum concentration of 10 μg/mL or higher of the anti-FKN antibody is achieved. In an alternative preferred embodiment of the present invention, the pharmaceutical composition of the present invention is formulated in such a manner that a serum concentration of 20 μg/mL or higher of the anti-FKN antibody is achieved.

In an alternative aspect, the present invention relates to a pharmaceutical composition comprising an anti-FKN antibody, wherein the pharmaceutical composition is used in such a manner that a therapeutically effective serum anti-FKN antibody concentration is achieved after a single administration or multiple administrations to a human subject. The pharmaceutical composition of the present invention is not particularly limited and can be typically in the form of a formulation for injection prepared for subcutaneous administration. In one embodiment, the pharmaceutical composition of the present invention is used such that the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher. In a preferred embodiment, the pharmaceutical composition of the present invention is used such that the mean trough concentration of the anti-FKN antibody is 10 μg/mL or higher or 20 μg/mL or higher.

In the present invention, the pharmacokinetic characteristics of a concentration-time curve, for example, a maximum serum concentration (Cₘₐₓ), a time to reach Cₘₐₓ (tₘₐₓ), and an area under the serum concentration-time curve (AUC) are examined by statistical approaches sufficiently established in the field of pharmacokinetics. When the ratio of a population mean of an evaluation parameter as described above between a test formulation and a standard formulation is 0.80 to 1.25, these formulations are generally regarded as being biologically equivalent.

In the present invention, mean values of the pharmacokinetic parameters such as Cₘₐₓ and AUC can be calculated by any of geometric mean and arithmetic mean methods. In the present specification, mean Cₘₐₓ, mean AUC, mean tFKN concentration, and mean trough concentration are indicated by arithmetic mean values, and tₘₐₓ is indicated by a median value. Even if a mean value of interest is calculated by a method different from that described in the present specification, the mean value is intended to belong to the scope of claims according to the present invention as long as the corresponding mean value calculated according to the method of the present specification falls within the numerical range described in the scope of claims.

In the present invention, the term “one dose” or “per dose” regarding the dose of the anti-FKN antibody refers to the absolute amount of the anti-FKN antibody per administration. Thus, for example, the term “200 mg per dose” may mean that one set of the pharmaceutical composition of the present invention comprising 200 mg of the anti-FKN antibody is administered, or may mean that two sets of the pharmaceutical composition of the present invention comprising 100 mg of the anti-FKN antibody are administered at the same time.

In the present invention, for example, the term “dosing intervals of once a week to once every two weeks” regarding the dosing interval of the anti-FKN antibody means that the dosing interval from the n^{th} (n represents an integer) administration to the (n+1)^{th} administration is 1 week to 2 weeks, and the dosing interval between the n^{th} administration and the (n+1)^{th} administration may be different from the dosing interval between the (n+1)^{th} administration and the (n+2)^{th} administration. For example, the term “dosing intervals of once a week to once every two weeks” naturally includes the case where the dosing interval from the first administration to the second administration is 1 week and the dosing interval from the second administration to the third administration is 2 weeks.

In the present specification, the term “human” or “human subject” means a healthy adult male, and typically, any human who has rheumatoid arthritis or manifests a clinical sign and symptom of any disease or disorder that may cause rheumatoid arthritis. In the present invention, the term “human” or “human subject” is preferably a rheumatic disease patient who has not obtained adequate therapeutic effects from at least one type of antirheumatic drug or anti-TNF formulation, has not obtained sustained therapeutic effects therefrom, or has not respond thereto.

The term “rheumatoid arthritis” refers to a disease state that can be diagnosed according to the 1987 ACR Classification Criteria or the 2010 ACR/EULAR Classification Criteria. Examples of physiological indexes of rheumatoid arthritis include symmetrical joint swelling and pain on passive movement, which are characteristics, but not unchangeable, of rheumatoid arthritis. A candidate for the treatment described in the present invention can be resistant to methotrexate or an anti-TNF formulation (adalimumab, infliximab, golimumab, certolizumab pegol, or etanercept) from the viewpoint that methotrexate or the anti-TNF formulation is not effective or not completely effective for the treatment of its symptom.

### 2. Anti-FKN Antibody

In the present invention, the term “anti-FKN antibody” refers to humanized anti-human fractalkine antibody H3-2L4 or an antibody functionally equivalent thereto. In the present invention, the “functionally equivalent antibody” refers to an antibody that is equivalent to the antibody H3-2L4 in terms of at least any of or preferably all of binding affinity, neutralizing activity, cross reactivity, and pharmacokinetics in blood against human FKN.

In the present invention, the term “anti-FKN antibody” may include an antigen-binding fragment thereof. Such an antigen-binding fragment is not particularly limited as long as the antigen-binding fragment is a functional and structural fragment of the anti-FKN antibody, maintains the binding activity of the antibody against FKN, and does not significantly differ in pharmacokinetics in blood from the complete antibody. Examples of the antigen-binding fragment of the antibody include, but are not limited to, Fab, Fab′, F(ab′)₂, Fv, single-chain (scFv), variants thereof, fusion proteins containing the antibody moiety, other modified structures of immunoglobulin molecules containing an antigen recognition site, and the like.

In one embodiment, the anti-FKN antibody of the present invention can be any antibody comprising the following CDR sequences:

(a) CDR-H1 comprising the amino acid sequence of SEQ ID NO: 15 (NYYIH);

(b) CDR-H2 comprising the amino acid sequence of SEQ ID NO: 16 (WIYPGDGSPKFNERFKG);

(c) CDR-H3 comprising the amino acid sequence of SEQ ID NO: 17 (GPTDGDYFDY);

(d) CDR-L1 comprising the amino acid sequence of SEQ ID NO: 18 (RASGNIHNFLA);

(e) CDR-L2 comprising the amino acid sequence of SEQ ID NO: 19 (NEKTLAD); and

(f) CDR-L3 comprising the amino acid sequence of SEQ ID NO: 20 (QQFWSTPYT).

In an alternative embodiment, the anti-FKN antibody can be an antibody comprising a heavy chain and a light chain, wherein a heavy chain variable region of the antibody comprises the amino acid sequence of SEQ ID NO: 21 (QVQLVQSGAEVKKPGASVKVSCKASGYTFTNYYIHWVKQAPGQGLEWIGWIYPGDG SPKFNERFKGRTTLTADKSTNTAYMLLSSLRSDDTAVYFCATGPTDGDYFDYWGQGT TVTVSS), SEQ ID NO:13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS), SEQ ID NO: 22 (QVQLVQSGAEVKKPGASVKVSCKASGYTFTNYYIHWVRQAPGQGLEWIGWIYPGDG SPKFNERFKGRTTLTRDKSTNTAYMELSSLRSDDTAVYFCATGPTDGDYFDYWGQGT TVTVSS), SEQ ID NO: 23 (QVQLVQSGAEVKKPGASVKVSCKASGYTFTNYYIHWVRQAPGQGLEWIGWIYPGDG SPKFNERFKGRTTMTADTSTSTAYMELSSLRSEDTAVYFCARGPTDGDYFDYWGQGT TVTVSS), or SEQ ID NO: 24 (QVQLVQSGAEVKKPGASVKVSCKASGYTFTNYYIHWVRQAPGQGLEWIGWIYPGDG SPKFNERFKGRTTLTADKSTSTAYMELSSLRSEDTAVYFCARGPTDGDYFDYWGQGT TVTVSS), and a light chain variable region of the antibody comprises the amino acid sequence of SEQ ID NO: 25 (DIQMTQSPSSLSASVGDRVTITCRASGNIHNFLAWYQQKPGKAPKFLVYNEKTLAD GVPSRFSGSGSGTQYTLTISSLQPEDFATYFCQQFWSTPYTFGGGTKVEIK), SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK), or SEQ ID NO: 26 (DIQMTQSPSSLSASVGDRVTITCRASGNIHNFLAWYQQKPGKAPKLLIYNEKTLAD GVPSRFSGSGSGTQYTLTISSLQPEDFATYFCQQFWSTPYTFGGGTKVEIK).

In a preferred embodiment, the anti-FKN antibody can be an antibody comprising a heavy chain and a light chain, wherein a heavy chain variable region of the antibody comprises the amino acid sequence of SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS) and a light chain variable region of the antibody comprises the amino acid sequence of SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK).

In a particular embodiment, the anti-FKN antibody is an antibody comprising a constant region of human IgG2 isotype.

In a particular embodiment, the anti-FKN antibody is an antibody wherein a Fc region of the constant region of human IgG2 isotype contains V234A and/or G237A mutations.

In a particularly preferred embodiment of the present invention, the anti-FKN antibody is antibody H3-2L4 consisting of a heavy chain consisting of the amino acid sequence represented by SEQ ID NO: 11 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSSASTKGPSVFPLAPCSRSTSESTAALGCLVKDYFPEPVTVSWNSGALTSGVH TFPAVLQSSGLYSLSSVVTVPSSNFGTQTYTCNVDHKPSNTKVDKTVERKCCVECPP CPAPPAAAPSVFLFPPKPKDTLMISRTPEVTCVVVDVSHEDPEVQFNWYVDGVEVHN AKTKPREEQFNSTFRVVSVLTVVHQDWLNGKEYKCKVSNKGLPAPIEKTISKTKGQP REPQVYTLPPSREEMTKNQVSLTCLVKGFYPSDIAVEWESNGQPENNYKTTPPMLDS DGSFFLYSKLTVDKSRWQQGNVFSCSVMHEALHNHYTQKSLSLSPGK) and a light chain consisting of the amino acid sequence represented by SEQ ID NO: 12 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

Needless to say, an antibody derived from any of the anti-FKN antibodies listed above by appropriate alteration (e.g., modification of the antibody or partial substitution, addition, or deletion of the amino acid sequence of the antibody) so as to maintain the functions of the antibody or in order to add or improve the functions of the antibody is also included in the antibody of the present invention. More specifically, an antibody lacking lysine (Lys) positioned at the carboxy terminus (C terminus) of the heavy chain by an artificial method such as genetic engineering in order to decrease heterogeneity among antibodies produced by antibody-producing cells is also included in the scope of the present invention. In addition, the anti-FKN antibody contained in the pharmaceutical composition of the present invention is not necessarily required to have complete homogeneity. For example, an anti-FKN antibody lacking lysine (Lys) positioned at the carboxy terminus (C terminus) of the heavy chain and an anti-FKN antibody that does not lack this lysine may coexist in the pharmaceutical composition of the present invention as long as the pharmaceutical composition of the present invention maintains the intended functions.

The anti-FKN antibody may be modified, if desired. The modification of the anti-FKN antibody may be a modification that changes (a) the three-dimensional structure of an amino acid sequence in a modification region, such as sheet or helix conformation; (b) the electric charge or hydrophobic status of the molecule at a target site; or (c) the effects of a modification on the maintenance of side chain volume, or may be a modification by which these changes are not clearly observed.

The modification of the anti-FKN antibody may be achieved by, for example, the substitution, deletion, and/or addition of a constituent amino acid residue(s).

In the present specification, the amino acid is used in the broadest sense thereof and includes not only natural amino acids, for example, serine (Ser), asparagine (Asn), valine (Val), leucine (Leu), isoleucine (Ile), alanine (Ala), tyrosine (Tyr), glycine (Gly), lysine (Lys), arginine (Arg), histidine (His), aspartic acid (Asp), glutamic acid (Glu), glutamine (Gln), threonine (Thr), cysteine (Cys), methionine (Met), phenylalanine (Phe), tryptophan (Trp), and proline (Pro) but non-natural amino acids such as amino acid variants and derivatives. Those skilled in the art should understand, by taking this wide definition into consideration, that examples of the amino acid in the present specification include: L-amino acids; D-amino acids; chemically modified amino acids such as amino acid variants and amino acid derivatives; amino acids, such as norleucine, β-alanine, and ornithine, which do not serve as materials constituting proteins in vivo; and chemically synthesized compounds having the characteristics of amino acids generally known to those skilled in the art. Examples of the non-natural amino acids include α-methylamino acids (α-methylalanine, etc.), D-amino acids (D-aspartic acid, D-glutamic acid, etc.), histidine-like amino acids (2-amino-histidine, β-hydroxy-histidine, homohistidine, α-fluoromethyl-histidine, α-methyl-histidine, etc.), amino acids having extra methylene in their side chains (“homo” amino acids), and amino acids in which a carboxylic acid functional group in the side chain is replaced with a sulfonic acid group (cysteic acid, etc.).

Naturally occurring amino acid residues can be classified into, for example, the following groups based on general side chain characteristics:
(1) hydrophobic residues: Met, Ala, Val, Leu, and Ile;
(2) neutral hydrophilic residues: Cys, Ser, and Thr;
(3) acidic residues: Asp and Glu;
(4) basic residues: Asn, Gln, His, Lys, and Arg;
(5) residues influencing chain orientation: Gly and Pro; and
(6) aromatic residues: Trp, Tyr, and Phe.

The non-conservative substitution of an amino acid sequence constituting the anti-FKN antibody may be performed by replacing an amino acid belonging to one of these groups with an amino acid belonging to any of the other groups. More conservative substitution may be performed by replacing an amino acid belonging to one of these groups with another amino acid belonging to the same group thereas. Likewise, the deletion or the substitution in an amino acid sequence may be appropriately performed.

### 3. Pharmaceutical Composition and Formulation

The pharmaceutical composition of the present invention comprises a therapeutically effective amount of the anti-FKN antibody. Such an amount of the anti-FKN antibody contained in the pharmaceutical composition of the present invention can vary according to the administration route and dosing interval of the pharmaceutical composition of the present invention.

In one embodiment of the present invention, the pharmaceutical composition of the present invention can comprise the anti-FKN antibody in an amount from 100 mg to 400 mg. In a particular embodiment of the present invention, the formulation of the present invention can comprise the anti-FKN antibody in an amount of at least 100 mg, at least 150 mg, at least 200 mg, at least 250 mg, at least 300 mg, at least 350 mg, or at least 400 mg. In a further alternative embodiment, the formulation of the present invention can comprise the anti-FKN antibody in an amount of 100 mg, 200 mg, or 400 mg.

The dosage form of the pharmaceutical composition of the present invention is not particularly limited and is typically a formulation for injection prepared for subcutaneous administration. The pharmaceutical composition of the present invention can be prepared, for example, as a formulation for injection of the anti-FKN antibody, together with a pharmaceutically acceptable excipient, in injectable water, physiological saline, or phosphate-buffered saline without limitations. Examples of the pharmaceutically acceptable excipient used in the present invention include, but are not limited to, a stabilizer, a surfactant, and a preservative.

The stabilizer used in the present invention is, for example, a carbohydrate, a saccharide, or a sugar (e.g., sucrose) accepted by an authority as an appropriate additive or excipient for pharmaceutical formulations. The concentration of the stabilizer is 15 to 250 mM, 150 to 250 mM, or 200 mM. The formulation may contain a secondary stabilizer.

Appropriate examples of the pharmaceutically acceptable surfactant used in the present invention include, but are not limited to, polyoxyethylene sorbitan fatty acid ester (Tween), polyethylene polypropylene glycol, polyoxyethylene stearate, polyoxyethylene alkyl ether (e.g., polyoxyethylene monolauryl ether), alkylphenyl polyoxyethylene ether (Triton-X), polyoxyethylene-polyoxypropylene copolymers (Poloxamer and Pluronic), and sodium dodecyl sulfate (SDS). The most appropriate polyoxyethylene sorbitan fatty acid esters are polysorbate 20 (Tween 20) and polysorbate 80 (Tween 80). The concentration of the surfactant is 0.01 to 0.1% (w/v), 0.01 to 0.08% (w/v), or 0.025 to 0.075% (w/v), for example, 0.05% (w/v).

Examples of the preservative used in the present invention includes, but are not limited to, paraben, benzyl alcohol, sodium benzoate, phenol, benzalkonium chloride, thimerosal, chlorobutanol, benzoic acid, sodium bisulfite, sodium propionate, and arbitrary combinations or mixtures thereof.

Examples of the buffer used in the present invention include, but are not limited to, histidine, citrate, phosphate, glycine, acetate, and arbitrary combinations or mixtures thereof.

The formulation of the present invention may contain a buffer or a pH adjuster for controlling pH. In one embodiment, the formulation of the present invention has a pH in the range of 4.0 to 9.0, in the range of 5.0 to 9.0, in the range of 5.0 to 8.0, in the range of 5.0 to 7.5, in the range of 5.5 to 7.0, or in the range of 5.5 to 6.5.

Those skilled in the art should understand that the formulation of the present invention can be isotonic to human blood, i.e., the formulation of the present invention has essentially the same osmotic pressure as that of human blood. Such an isotonic formulation generally has an osmotic pressure of 250 mOSm to 350 mOSm. The isotonicity can be measured by use of, for example, a vapor pressure or ice freezing-type osmometer. Examples of the tonicity agent used in the present invention include, but are not limited to, saccharides, salts, and amino acids.

In addition to the excipients mentioned above, typical excipients and processes for the production of the formulation for injection for subcutaneous administration are known in the art. See, for example,

Introduction to Pharmaceutical Dosage Forms, 1985, Ansel, H. C., Lea and Febiger, Philadelphia, Pa.; Remington's Pharmaceutical Sciences, 1995, Mack Publ. Co., Easton, Pa. This literature is incorporated herein by reference in its entirety.

In one embodiment of the present invention, the pharmaceutical composition of the present invention can be any of

a pharmaceutical composition for subcutaneous administration formulated such that,

when 100 mg of the anti-fractalkine antibody is subcutaneously administered by a single administration to a human,

mean Cₘₐₓ is a value that falls within the numerical range of 80% to 125% of 10 μg/mL,

mean AUC_{(0-336 h)} is a value that falls within the numerical range of 80% to 125% of 2.4×10³ μg·h/mL, or

mean AUC₍₀₋ₜ₎ is a value that falls within the numerical range of 80% to 125% of 3.0×10³ μg·h/mL;

a pharmaceutical composition for subcutaneous administration formulated such that

when 200 mg of the anti-fractalkine antibody is subcutaneously administered by a single administration to a human,

mean Cₘₐₓ is a value that falls within the numerical range of 80% to 125% of 27 μg/mL,

mean AUC_{(0-336 h)} is a value that falls within the numerical range of 80% to 125% of 7.4×10³ μg·h/mL, or

mean AUC₍₀₋ₜ₎ is a value that falls within the numerical range of 80% to 125% of 1.2×10⁴ μg·h/mL; and

a pharmaceutical composition for subcutaneous administration formulated such that

when 400 mg of the anti-fractalkine antibody is subcutaneously administered by a single administration to a human,

mean Cₘₐₓ is a value that falls within the numerical range of 80% to 125% of 43 μg/mL,

mean AUC_{(0-336 h)} is a value that falls within the numerical range of 80% to 125% of 1.2×10⁴ μg·h/mL, or

mean AUC₍₀₋ₜ₎ is a value that falls within the numerical range of 80% to 125% of 2.7×10⁴ μg·h/mL.

In a preferred embodiment of the present invention, the anti-fractalkine antibody is antibody H3-2L4 consisting of a heavy chain consisting of the amino acid sequence represented by SEQ ID NO: 11 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSSASTKGPSVFPLAPCSRSTSESTAALGCLVKDYFPEPVTVSWNSGALTSGVH TFPAVLQSSGLYSLSSVVTVPSSNFGTQTYTCNVDHKPSNTKVDKTVERKCCVECPP CPAPPAAAPSVFLFPPKPKDTLMISRTPEVTCVVVDVSHEDPEVQFNWYVDGVEVHN AKTKPREEQFNSTFRVVSVLTVVHQDWLNGKEYKCKVSNKGLPAPIEKTISKTKGQP REPQVYTLPPSREEMTKNQVSLTCLVKGFYPSDIAVEWESNGQPENNYKTTPPMLDS DGSFFLYSKLTVDKSRWQQGNVFSCSVMHEALHNHYTQKSLSLSPGK) and a light chain consisting of the amino acid sequence represented by SEQ ID NO: 12 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

### 4. Dose, Dosing Interval, and the Number of Doses

The pharmaceutical composition of the present invention is administered to a human subject at a dose that brings about therapeutically effective amelioration in rheumatoid arthritis. Thus, the pharmaceutical composition of the present invention is administered such that the serum concentration or the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher. Preferably, the pharmaceutical composition of the present invention is administered by multiple administrations such that the mean trough concentration of the anti-FKN antibody is 10 μg/mL or higher or 20 μg/mL or higher.

In one embodiment of the present invention, the pharmaceutical composition of the present invention is used such that at least 100 mg per dose of the anti-fractalkine antibody is subcutaneously administered to a human. In an alternative embodiment of the present invention, the pharmaceutical composition of the present invention is used such that 100 mg to 400 mg per dose of the anti-fractalkine antibody is subcutaneously administered to a human. In one embodiment of the present invention, the pharmaceutical composition of the present invention is used such that 100 mg, 200 mg, or 400 mg per dose of the anti-fractalkine antibody is subcutaneously administered to a human. In a preferred embodiment of the present invention, the pharmaceutical composition of the present invention is used such that 200 mg to 400 mg per dose of the anti-fractalkine antibody is subcutaneously administered to a human. In an alternative preferred embodiment of the present invention, the pharmaceutical composition of the present invention is used such that 200 mg or 400 mg per dose of the anti-fractalkine antibody is subcutaneously administered to a human.

The number of doses and dosing interval of the pharmaceutical composition of the present invention can vary according to the amount of the anti-FKN antibody administered per dose and an administration route, etc.

In one embodiment of the present invention, the pharmaceutical composition of the present invention is used such that at least 100 mg per dose of the anti-FKN antibody is subcutaneously administered by multiple administrations to a human subject in need thereof at dosing intervals of once a week to once every two months. In an alternative embodiment of the present invention, the pharmaceutical composition of the present invention is used such that at least 100 mg per dose of the anti-FKN antibody is subcutaneously administered by multiple administrations to a human subject in need thereof at dosing intervals of once a week to once a month. In an alternative embodiment of the present invention, the pharmaceutical composition of the present invention is used such that at least 100 mg per dose of the anti-FKN antibody is subcutaneously administered by multiple administrations to a human subject in need thereof at dosing intervals of once a week to once every two weeks. In a particular embodiment of the present invention, the pharmaceutical composition of the present invention is used such that at least 100 mg per dose of the anti-FKN antibody is subcutaneously administered at dosing intervals of every other week after two once-a-week administrations.

In one embodiment of the present invention, the pharmaceutical composition of the present invention is used such that 100 mg to 400 mg per dose of the anti-FKN antibody is subcutaneously administered by multiple administrations to a human subject in need thereof at dosing intervals of once a week to once every two months. In an alternative embodiment of the present invention, the pharmaceutical composition of the present invention is used such that 100 mg to 400 mg per dose of the anti-FKN antibody is subcutaneously administered by multiple administrations to a human subject in need thereof at dosing intervals of once a week to once a month. In an alternative embodiment of the present invention, the pharmaceutical composition of the present invention is used such that 100 mg to 400 mg per dose of the anti-FKN antibody is subcutaneously administered by multiple administrations to a human subject in need thereof at dosing intervals of once a week to once every two weeks. In a particular embodiment of the present invention, the pharmaceutical composition of the present invention is used such that 100 mg to 400 mg per dose of the anti-FKN antibody is subcutaneously administered at dosing intervals of every other week after two once-a-week administrations.

In an alternative embodiment of the present invention, the pharmaceutical composition of the present invention is used such that 100 mg, 200 mg, or 400 mg per dose of the anti-FKN antibody is subcutaneously administered by multiple administrations to a human subject in need thereof at dosing intervals of once a week to once every two months. In an alternative embodiment of the present invention, the pharmaceutical composition of the present invention is used such that 100 mg, 200 mg, or 400 mg per dose of the anti-FKN antibody is subcutaneously administered by multiple administrations to a human subject in need thereof at dosing intervals of once a week to once a month. In an alternative embodiment of the present invention, the pharmaceutical composition of the present invention is used such that 100 mg, 200 mg, or 400 mg per dose of the anti-FKN antibody is subcutaneously administered by multiple administrations to a human subject in need thereof at dosing intervals of once a week to once every two weeks. In a particular embodiment of the present invention, the pharmaceutical composition of the present invention is used such that 100 mg, 200 mg, or 400 mg per dose of the anti-FKN antibody is subcutaneously administered at dosing intervals of every other week after two once-a-week administrations.

In an alternative embodiment of the present invention, the pharmaceutical composition of the present invention is used such that 100 mg to 200 mg per dose of the anti-FKN antibody is subcutaneously administered by multiple administrations to a human subject in need thereof at dosing intervals of once a week to once every two months. In an alternative embodiment of the present invention, the pharmaceutical composition of the present invention is used such that 100 mg to 200 mg per dose of the anti-FKN antibody is subcutaneously administered by multiple administrations to a human subject in need thereof at dosing intervals of once a week to once a month. In an alternative embodiment of the present invention, the pharmaceutical composition of the present invention is used such that 100 mg to 200 mg per dose of the anti-FKN antibody is subcutaneously administered by multiple administrations to a human subject in need thereof at dosing intervals of once a week to once every two weeks. In a particular embodiment of the present invention, the pharmaceutical composition of the present invention is used such that 100 mg to 200 mg per dose of the anti-FKN antibody is subcutaneously administered at dosing intervals of every other week after two once-a-week administrations.

In a preferred embodiment of the present invention, the pharmaceutical composition of the present invention is used such that 200 mg to 400 mg per dose of the anti-FKN antibody is subcutaneously administered by multiple administrations to a human subject in need thereof at dosing intervals of once a week to once every two months. In an alternative preferred embodiment of the present invention, the pharmaceutical composition of the present invention is used such that 200 mg to 400 mg per dose of the anti-FKN antibody is subcutaneously administered by multiple administrations to a human subject in need thereof at dosing intervals of once a week to once a month. In an alternative preferred embodiment of the present invention, the pharmaceutical composition of the present invention is used such that 200 mg to 400 mg per dose of the anti-FKN antibody is subcutaneously administered by multiple administrations to a human subject in need thereof at dosing intervals of once a week to once every two weeks. In an alternative preferred embodiment of the present invention, the pharmaceutical composition of the present invention is used such that 200 mg to 400 mg per dose of the anti-FKN antibody is subcutaneously administered at dosing intervals of every other week after two once-a-week administrations.

The number of doses of the pharmaceutical composition of the present invention is not particularly limited as long as the pharmaceutical composition of the present invention brings about therapeutically effective amelioration in rheumatoid arthritis. The number of doses of the pharmaceutical composition of the present invention can vary according to the amount of the anti-FKN antibody administered per dose, an administration route, and dosing intervals.

In one embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once every two months.

In an alternative embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once a month.

In a further alternative embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once every two weeks.

In a particular embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of every other week after two once-a-week administrations.

In a particular embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once every two months such that the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher.

In a further alternative embodiment of the present invention, the pharmaceutical composition of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once a month such that the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher.

In a further alternative embodiment of the present invention, the pharmaceutical composition or the anti-FKN antibody of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of once a week to once every two weeks such that the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher.

In a further alternative embodiment of the present invention, the pharmaceutical composition or the anti-FKN antibody of the present invention can be subcutaneously administered at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times or more at dosing intervals of every other week after two once-a-week administrations such that the mean trough concentration of the anti-FKN antibody is 5 μg/mL or higher, 10 μg/mL or higher, 15 μg/mL or higher, 20 μg/mL or higher, 25 μg/mL or higher, 30 μg/mL or higher, 35 μg/mL or higher, or 40 μg/mL or higher.

Hereinafter, particular aspects of the present invention will be illustrated without limitations.

In one aspect, the present invention relates to a pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the pharmaceutical composition is used such that at least 100 mg per dose of the anti-fractalkine antibody is subcutaneously administered to a human.

In an alternative aspect, the present invention relates to a pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the pharmaceutical composition is used such that 100 mg to 400 mg per dose of the anti-fractalkine antibody is subcutaneously administered by multiple administrations to a human.

In an alternative aspect, the present invention relates to a pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the pharmaceutical composition is used such that 100 mg to 200 mg per dose of the anti-fractalkine antibody is subcutaneously administered by multiple administrations to a human.

In a further alternative aspect, the present invention relates to a pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region contains V234A and G237A mutations, and the pharmaceutical composition is used such that 200 mg to 400 mg per dose of the anti-fractalkine antibody is subcutaneously administered by multiple administrations to a human.

In a further alternative aspect, the present invention relates to a pharmaceutical composition for the treatment of rheumatoid arthritis,

the pharmaceutical composition comprising an anti-fractalkine antibody and a pharmaceutically acceptable excipient, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the pharmaceutical composition is used such that 100 mg, 200 mg, or 400 mg per dose of the anti-fractalkine antibody is subcutaneously administered by multiple administrations to a human.

The pharmaceutical composition of the present invention is subcutaneously administered by multiple administrations at dosing intervals of once a week to once every two weeks to a human in such a manner that a mean trough concentration of 10 μg/mL or higher of the anti-FKN antibody is achieved. In a preferred embodiment, the pharmaceutical composition of the present invention is subcutaneously administered by multiple administrations at dosing intervals of once a week to once every two weeks to a human in such a manner that a mean trough concentration of 15 μg/mL or higher of the anti-FKN antibody is achieved. In a more preferred embodiment, the pharmaceutical composition of the present invention is subcutaneously administered by multiple administrations at dosing intervals of once a week to once every two weeks to a human in such a manner that a mean trough concentration of 20 μg/mL or higher of the anti-FKN antibody is achieved.

In a preferred embodiment, the pharmaceutical composition of the present invention is a pharmaceutical composition for subcutaneous administration formulated such that when 100 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 10 μg/mL;

mean AUC_{(0-336 h)} is a value that falls within the numerical range of 80% to 125% of 2.4×10³ μg·h/mL; and

mean AUC₍₀₋ₜ₎ is a value that falls within the numerical range of 80% to 125% of 3.0×10³ μg·h/mL.

In an alternative preferred embodiment, the pharmaceutical composition of the present invention is a pharmaceutical composition for subcutaneous administration formulated such that when 200 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 27 μg/mL;

mean AUC_{(0-336 h)} is a value that falls within the numerical range of 80% to 125% of 7.4×10³ μg·h/mL; and

mean AUC₍₀₋ₜ₎ is a value that falls within the numerical range of 80% to 125% of 1.2×10⁴ μg·h/mL.

In a further alternative preferred embodiment, the pharmaceutical composition of the present invention is a pharmaceutical composition for subcutaneous administration formulated such that when 400 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 43 μg/mL;

mean AUC_{(0-336 h)} is a value that falls within the numerical range of 80% to 125% of 1.2×10⁴ μg·h/mL; and

mean AUC₍₀₋ₜ₎ is a value that falls within the numerical range of 80% to 125% of 2.7×10⁴ μg·h/mL.

In an alternative aspect, the present invention relates to a pharmaceutical composition for use in a method for treating rheumatoid arthritis,

the treatment method comprising subcutaneously administering a therapeutically effective amount of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the therapeutically effective amount is, when the pharmaceutical composition comprising the anti-fractalkine antibody is subcutaneously administered by a single administration, a dose that brings about any one, two, or three of the following pharmacokinetic parameters:

mean Cₘₐₓ is a value that falls within the numerical range of 80% to 125% of 10 μg/mL;

mean AUC_{(0-336 h)} is a value that falls within the numerical range of 80% to 125% of 2.4×10³ μg·h/mL; and

mean AUC₍₀₋ₜ₎ is a value that falls within the numerical range of 80% to 125% of 3.0×10³ μg·h/mL.

In an alternative aspect, the present invention relates to a pharmaceutical composition for use in a method for treating rheumatoid arthritis,

the treatment method comprising subcutaneously administering a therapeutically effective amount of an anti-fractalkine antibody to a human in need thereof,

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the therapeutically effective amount is, when the pharmaceutical composition comprising the anti-fractalkine antibody is subcutaneously administered by a single administration, a dose that brings about any one, two, or three of the following pharmacokinetic parameters:

mean Cₘₐₓ is a value that falls within the numerical range of 80% to 125% of 27 μg/mL;

mean AUC_{(0-336 h)} is a value that falls within the numerical range of 80% to 125% of 7.4×10³ μg·h/mL; and

mean AUC₍₀₋ₜ₎ is a value that falls within the numerical range of 80% to 125% of 1.2×10⁴ μg·h/mL.

In an alternative aspect, the present invention relates to a pharmaceutical composition for use in a method for treating rheumatoid arthritis,

the treatment method comprising subcutaneously administering a therapeutically effective amount of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

the therapeutically effective amount is, when the pharmaceutical composition comprising the anti-fractalkine antibody is subcutaneously administered by a single administration, a dose that brings about any one, two, or three of the following pharmacokinetic parameters:

mean Cₘₐₓ is a value that falls within the numerical range of 80% to 125% of 43 μg/mL;

mean AUC_{(0-336 h)} is a value that falls within the numerical range of 80% to 125% of 1.2×10⁴ μg·h/mL; and

mean AUC₍₀₋ₜ₎ is a value that falls within the numerical range of 80% to 125% of 2.7×10⁴ μg·h/mL.

In a preferred embodiment, the anti-fractalkine antibody is antibody H3-2L4 consisting of a heavy chain consisting of the amino acid sequence represented by SEQ ID NO: 11 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSSASTKGPSVFPLAPCSRSTSESTAALGCLVKDYFPEPVTVSWNSGALTSGVH TFPAVLQSSGLYSLSSVVTVPSSNFGTQTYTCNVDHKPSNTKVDKTVERKCCVECPP CPAPPAAAPSVFLFPPKPKDTLMISRTPEVTCVVVDVSHEDPEVQFNWYVDGVEVHN AKTKPREEQFNSTFRVVSVLTVVHQDWLNGKEYKCKVSNKGLPAPIEKTISKTKGQP REPQVYTLPPSREEMTKNQVSLTCLVKGFYPSDIAVEWESNGQPENNYKTTPPMLDS DGSFFLYSKLTVDKSRWQQGNVFSCSVMHEALHNHYTQKSLSLSPGK) and a light chain consisting of the amino acid sequence represented by SEQ ID NO: 12 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

In a preferred embodiment, the pharmaceutical composition is a pharmaceutical composition comprising 100 mg, 200 mg, or 400 mg of the anti-fractalkine antibody.

In a preferred embodiment, the pharmaceutical composition is a pharmaceutical composition that is used such that 100 mg, 200 mg, or 400 mg per dose of the anti-fractalkine antibody is subcutaneously administered.

In a preferred embodiment, the pharmaceutical composition is a pharmaceutical composition that is subcutaneously administered by multiple administrations at dosing intervals of once a week to once every two weeks to a human.

In a preferred embodiment, the pharmaceutical composition is a pharmaceutical composition that is subcutaneously administered such that the mean trough concentration of the anti-fractalkine antibody is 10 μg/mL or higher.

In a preferred embodiment, the pharmaceutical composition is a pharmaceutical composition that is subcutaneously administered such that the mean trough concentration of the anti-fractalkine antibody is 20 μg/mL or higher.

In an alternative aspect, the present invention relates to a therapeutic agent for rheumatoid arthritis, comprising an anti-fractalkine antibody, wherein

the therapeutic agent for rheumatoid arthritis is used such that 100 mg to 400 mg of the anti-fractalkine antibody is subcutaneously administered at dosing intervals of once a week to once every two weeks to a human, and

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a therapeutic agent for rheumatoid arthritis, comprising an anti-fractalkine antibody, wherein

the therapeutic agent for rheumatoid arthritis is used such that 100 mg to 200 mg of the anti-fractalkine antibody is subcutaneously administered at dosing intervals of once a week to once every two weeks to a human, and

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a therapeutic agent for rheumatoid arthritis, comprising an anti-fractalkine antibody, wherein

the therapeutic agent for rheumatoid arthritis is used such that 200 mg to 400 mg of the anti-fractalkine antibody is subcutaneously administered at dosing intervals of once a week to once every two weeks to a human, and

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a therapeutic agent for rheumatoid arthritis, comprising an anti-fractalkine antibody, wherein

the therapeutic agent for rheumatoid arthritis is used such that 100 mg, 200 mg, or 400 mg of the anti-fractalkine antibody is subcutaneously administered at dosing intervals of once a week to once every two weeks to a human, and

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In a preferred embodiment, the anti-fractalkine antibody is antibody H3-2L4 consisting of a heavy chain consisting of the amino acid sequence represented by SEQ ID NO: 11 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSSASTKGPSVFPLAPCSRSTSESTAALGCLVKDYFPEPVTVSWNSGALTSGVH TFPAVLQSSGLYSLSSVVTVPSSNFGTQTYTCNVDHKPSNTKVDKTVERKCCVECPP CPAPPAAAPSVFLFPPKPKDTLMISRTPEVTCVVVDVSHEDPEVQFNWYVDGVEVHN AKTKPREEQFNSTFRVVSVLTVVHQDWLNGKEYKCKVSNKGLPAPIEKTISKTKGQP REPQVYTLPPSREEMTKNQVSLTCLVKGFYPSDIAVEWESNGQPENNYKTTPPMLDS DGSFFLYSKLTVDKSRWQQGNVFSCSVMHEALHNHYTQKSLSLSPGK) and a light chain consisting of the amino acid sequence represented by SEQ ID NO: 12 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

In one embodiment, the present invention relates to the pharmaceutical composition comprising the anti-fractalkine antibody, wherein the pharmaceutical composition is subcutaneously administered such that the mean serum total FKN concentration at the steady state is 60 μg/mL or higher, 70 μg/mL or higher, 80 μg/mL or higher, 90 μg/mL or higher, 100 μg/mL or higher, 110 μg/mL or higher, 120 μg/mL or higher, 130 μg/mL or higher, 140 μg/mL or higher, 150 μg/mL or higher, or 160 μg/mL or higher.

In one embodiment of the present invention, the pharmaceutical composition of the present invention is a pharmaceutical composition for subcutaneous administration, wherein when 100 mg of the anti-fractalkine antibody is administered by multiple administrations to a human, the mean serum total FKN concentration at the steady state is 70 μg/mL to 100 μg/mL.

In one embodiment of the present invention, the pharmaceutical composition of the present invention is a pharmaceutical composition for subcutaneous administration, wherein when the pharmaceutical composition is used such that 100 mg of the anti-fractalkine antibody is subcutaneously administered at dosing intervals of once a week to once every two weeks to a human, the mean serum total FKN concentration at the steady state is 70 μg/mL to 100 μg/mL.

In one embodiment of the present invention, the pharmaceutical composition of the present invention is a pharmaceutical composition for subcutaneous administration, wherein when 200 mg of the anti-fractalkine antibody is administered by multiple administrations to a human, the mean serum total FKN concentration at the steady state is 160 μg/mL to 190 μg/mL.

In one embodiment of the present invention, the pharmaceutical composition of the present invention is a pharmaceutical composition for subcutaneous administration, wherein when the pharmaceutical composition is used such that 200 mg of the anti-fractalkine antibody is subcutaneously administered at dosing intervals of once a week to once every two weeks to a human, the mean serum total FKN concentration at the steady state is 160 μg/mL to 190 μg/mL.

In one embodiment of the present invention, the pharmaceutical composition of the present invention is a pharmaceutical composition for subcutaneous administration, wherein when 400 mg of the anti-fractalkine antibody is administered by multiple administrations to a human, the mean serum total FKN concentration at the steady state is 380 μg/mL to 480 μg/mL.

In one embodiment of the present invention, the pharmaceutical composition of the present invention is a pharmaceutical composition for subcutaneous administration, wherein when the pharmaceutical composition is used such that 400 mg of the anti-fractalkine antibody is subcutaneously administered at dosing intervals of once a week to once every two weeks to a human, the mean serum total FKN concentration at the steady state is 380 μg/mL to 480 μg/mL.

In an alternative aspect, the present invention relates to a method for treating rheumatoid arthritis, the method comprising subcutaneously administering at least 100 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a method for treating rheumatoid arthritis,

the method comprising subcutaneously administering 100 to 400 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a method for treating rheumatoid arthritis,

the method comprising subcutaneously administering 100 to 200 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a method for treating rheumatoid arthritis,

the method comprising subcutaneously administering 200 to 400 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In one embodiment, the present invention relates to a method for treating rheumatoid arthritis,

the method comprising subcutaneously administering 100 mg, 200 mg, or 400 mg per dose of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations.

In the treatment method of the present invention, the anti-fractalkine antibody is subcutaneously administered by multiple administrations at dosing intervals of once a week to once every two weeks to a human in such a manner that a mean trough concentration of 10 μg/mL or higher of the anti-fractalkine antibody is achieved. In a preferred embodiment, the anti-fractalkine antibody in the treatment method of the present invention is subcutaneously administered by multiple administrations at dosing intervals of once a week to once every two weeks to a human in such a manner that a mean trough concentration of 15 μg/mL or higher of the anti-fractalkine antibody is achieved. In a more preferred embodiment, the anti-fractalkine antibody in the treatment method of the present invention is subcutaneously administered by multiple administrations at dosing intervals of once a week to once every two weeks to a human in such a manner that a mean trough concentration of 20 μg/mL or higher of the anti-fractalkine antibody is achieved.

In a preferred embodiment, the anti-fractalkine antibody is administered by a pharmaceutical composition for subcutaneous administration formulated such that when 100 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 10 μg/mL;

mean AUC_{(0-336 h)} is a value that falls within the numerical range of 80% to 125% of 2.4×10³ μg·h/mL; and

mean AUC₍₀₋ₜ₎ is a value that falls within the numerical range of 80% to 125% of 3.0×10³ μg·h/mL.

In an alternative preferred embodiment, the anti-fractalkine antibody is administered by a pharmaceutical composition for subcutaneous administration formulated such that when 200 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 27 μg/mL;

mean AUC_{(0-336 h)} is a value that falls within the numerical range of 80% to 125% of 7.4×10³ μg·h/mL; and

mean AUC₍₀₋ₜ₎ is a value that falls within the numerical range of 80% to 125% of 1.2×10⁴ μg·h/mL.

In a further alternative preferred embodiment, the anti-fractalkine antibody is administered by a pharmaceutical composition for subcutaneous administration formulated such that when 400 mg of the anti-fractalkine antibody is subcutaneously administered by a single administration to a human, any one, two, or three of the following pharmacokinetic parameters are brought about:

mean Cₘₐₓ is a value that falls within the numerical range of 80% to 125% of 43 μg/mL;

mean AUC_{(0-336 h)} is a value that falls within the numerical range of 80% to 125% of 1.2×10⁴ μg·h/mL; and

mean AUC₍₀₋ₜ₎ is a value that falls within the numerical range of 80% to 125% of 2.7×10⁴ μg·h/mL.

In an alternative aspect, the present invention relates to a method for treating rheumatoid arthritis,

the treatment method comprising subcutaneously administering a therapeutically effective amount of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

when a pharmaceutical composition comprising the anti-fractalkine antibody is subcutaneously administered by a single administration,

the therapeutically effective amount is a dose that brings about any one, two, or three of the following pharmacokinetic parameters:

mean Cₘₐₓ is a value that falls within the numerical range of 80% to 125% of 10 μg/mL;

mean AUC_{(0-336 h)} is a value that falls within the numerical range of 80% to 125% of 2.4×10³ μg·h/mL; and

mean AUC₍₀₋ₜ₎ is a value that falls within the numerical range of 80% to 125% of 3.0×10³ μg·h/mL.

In an alternative aspect, the present invention relates to a method for treating rheumatoid arthritis,

the treatment method comprising subcutaneously administering a therapeutically effective amount of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

when a pharmaceutical composition comprising the anti-fractalkine antibody is subcutaneously administered by a single administration,

the therapeutically effective amount is a dose that brings about any one, two, or three of the following pharmacokinetic parameters:

mean Cₘₐₓ is a value that falls within the numerical range of 80% to 125% of 27 μg/mL;

mean AUC_{(0-336 h)} is a value that falls within the numerical range of 80% to 125% of 7.4×10³ μg·h/mL; and

mean AUC₍₀₋ₜ₎ is a value that falls within the numerical range of 80% to 125% of 1.2×10⁴ μg·h/mL.

In an alternative aspect, the present invention relates to a method for treating rheumatoid arthritis,

the treatment method comprising subcutaneously administering a therapeutically effective amount of an anti-fractalkine antibody to a human in need thereof, wherein

the anti-fractalkine antibody is an antibody wherein:

a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSS);

a light chain variable region thereof comprises the amino acid sequence represented by SEQ ID NO: 14 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIK);

the antibody comprises a constant region of human IgG2 isotype; and

a Fc region of the constant region of human IgG2 isotype contains V234A and G237A mutations, and

when a pharmaceutical composition comprising the anti-fractalkine antibody is subcutaneously administered by a single administration,

the therapeutically effective amount is a dose that brings about any one, two, or three of the following pharmacokinetic parameters:

mean Cₘₐₓ is a value that falls within the numerical range of 80% to 125% of 43 μg/mL;

mean AUC_{(0-336 h)} is a value that falls within the numerical range of 80% to 125% of 1.2×10⁴ μg·h/mL; and

mean AUC₍₀₋ₜ₎ is a value that falls within the numerical range of 80% to 125% of 2.7×10⁴ μg·h/mL.

In a preferred embodiment, the anti-fractalkine antibody is antibody H3-2L4 consisting of a heavy chain consisting of the amino acid sequence represented by SEQ ID NO: 11 (QVQLVQSGAEVKKPGASVKVSCKASGYTFTNYYIHWVKQAPGQGLEWIGWIYPGDG SPKFNERFKGRTTLTADKSTNTAYMLLSSLRSEDTAVYFCATGPTDGDYFDYWGQGT TVTVSSASTKGPSVFPLAPCSRSTSESTAALGCLVKDYFPEPVTVSWNSGALTSGVH TFPAVLQSSGLYSLSSVVTVPSSNFGTQTYTCNVDHKPSNTKVDKTVERKCCVECPP CPAPPAAAPSVFLFPPKPKDTLMISRTPEVTCVVVDVSHEDPEVQFNWYVDGVEVHN AKTKPREEQFNSTFRVVSVLTVVHQDWLNGKEYKCKVSNKGLPAPIEKTISKTKGQP REPQVYTLPPSREEMTKNQVSLTCLVKGFYPSDIAVEWESNGQPENNYKTTPPMLDS DGSFFLYSKLTVDKSRWQQGNVFSCSVMHEALHNHYTQKSLSLSPGK) and a light chain consisting of the amino acid sequence represented by SEQ ID NO: 12 (DIQMTQSPSSLSASVGDRVTITCRASGNIHNFLAWYQQKPGKAPKLLIYNEKTLAD GVPSRFSGSGSGTDYTLTISSLQPEDFATYFCQQFWSTPYTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

In a preferred embodiment, in the treatment method of the present invention, 100 mg, 200 mg, or 400 mg per dose of the anti-fractalkine antibody is subcutaneously administered to a human.

In a preferred embodiment, in the treatment method of the present invention, the anti-fractalkine antibody is subcutaneously administered by multiple administrations at dosing intervals of once a week to once every two weeks to a human.

In a preferred embodiment, in the treatment method of the present invention, the anti-fractalkine antibody is subcutaneously administered such that the mean trough concentration of the anti-fractalkine antibody is 10 μg/mL or higher.

In a preferred embodiment, in the treatment method of the present invention, the anti-fractalkine antibody is subcutaneously administered such that the mean trough concentration of the anti-fractalkine antibody is 20 μg/mL or higher.

In one embodiment of the present invention, the human is a rheumatic disease patient who has not obtained adequate effects from at least one type of antirheumatic drug or anti-TNF formulation, has not obtained sustained effects therefrom, or has not respond thereto.

Those skilled in the art should understand that the present invention may be carried out by any one of or appropriate combination of two or more of all aspects described in the present specification unless a technical contradiction arises. Further, those skilled in the art should understood that the present invention can be preferably carried out by an appropriate combination of all preferred or advantageous aspects described in the present specification unless a technical contradiction arises.

Literatures cited in the present specification should be interpreted as being incorporated herein by reference in their entirety. Those skilled in the art can understand related contents disclosed in these literatures by reference as a part of the present specification without departing from the spirit and scope of the present invention according to the context of the present specification.

Literatures cited in the present specification are provided merely for the purpose of disclosing related techniques before the filing date of the present application. It should not be understood that the present inventors admit to having no right preceding such disclosure due to the prior inventions or any other reasons. All statements of these literatures are based on information which has been available by the present applicant, and there is no admission that the contents of these statements are accurate.

The terms in the present specification are used for illustrating particular embodiments and are not intended to limit the invention.

The term “comprise” or “include” used in the present specification means that described items (members, steps, factors, numbers, etc.) are present and the presence of the other items (members, steps, factors, numbers, etc.) is not excluded therefrom, unless the context evidently requires different interpretation. The term “consist of” encompasses aspects described by the terms “consist of” and/or “consist essentially of”.

All terms (including technical terms and scientific terms) used herein have the same meanings as those understood in a broad sense by those skilled in the art to which the present invention belongs, unless otherwise defined. The terms used herein should be interpreted as having meanings consistent with meanings in the present specification and related technical fields and should not be interpreted in an idealized or excessively formal sense, unless otherwise defined.

Terms such as “first” or “second” are used for expressing various factors. However, these factors are understood to be not limited by these terms themselves. These terms are used merely for differentiating one factor from the other factors. For example, the first factor may be described as the second factor, and vice versa, without departing from the scope of the present invention.

In the present specification, it should be understood that numerical values used for indicating component contents, numerical ranges, etc., are modified with the term “approximately” unless otherwise specified. For example, “10 μg” is interpreted as meaning “approximately 10 μg” unless otherwise specified. Those skilled in the art can naturally understand the extent thereof rationally according to the technical common sense and the context of the present specification.

It should be understood that each aspect indicated in a singular form used in the present specification and claims may be in a plural form, and vice versa, unless the context evidently requires different interpretation and unless a technical contradiction arises.

Hereinafter, the present invention will be described in more detail with reference to Examples. However, the present invention can be embodied by various aspects and is not intended to be limited by Examples described herein. Those skilled in the art can implement the present invention by various changes or modifications, additions, deletions, substitutions, etc., without departing from the spirit or scope of the present invention.

Abbreviations used in Examples are idiomatic abbreviations well known to those skilled in the art. Some of the abbreviations are shown below:
AUC: area under the serum concentration-time curve
AUC_{(0-inf)}: area under the serum concentration-time curve from zero time extrap-olated to infinite time
AUC₍₀₋ₜ₎ : area under the concentration-time curve from zero time to time of last quantifiable concentration
AUC_{(0-336 h)}: area under the concentration-time curve from zero (predose) to fixed time-point 336 h (2 weeks) after the end of infusion
CDAI: clinical disease activity index
CL: total clearance
CL/F: apparent clearance
CRP: C-reactive protein
Cₘₐₓ: maximum serum concentration
DNCₘₐₓ: dose-normalized Cₘₐₓ
DNAUC: dose-normalized AUC
ELISA: enzyme-linked immunosorbent assay
FKN: fractalkine
IgG: immunoglobulin G
NONMEN: nonlinear mixed effect model
SAS: statistical analysis system
SDAI: simple disease activity index
t_{1/2}: serum elimination half-life
tₘₐₓ: time to reach peak serum concentration
V_{d}: volume of distribution
V_{z}/F: apparent volume of distribution at terminal phase

### EXAMPLES

### Example 1

### Preparation of Humanized Anti-Human Fractalkine Antibody

Administration tests to humans given below employed the humanized anti-human fractalkine antibody H3-2L4. The preparation of H3-2L4, including humanization, was carried out as described in WO2011/052799. H3-2L4 used in Example 2 or later was prepared by methods described below in 1-1 and 1-2.

### 1-1. Expression Vector

A humanized anti-human fractalkine antibody (H3-2L4) expression vector was prepared as follows.

First, a signal sequence (SEQ ID NO: 3) was added to the N terminus of the amino acid sequence (SEQ ID NO: 1) of the heavy chain variable region (H3-2) of the humanized anti-human fractalkine antibody (H3-2L4), and the amino acid sequence (SEQ ID NO: 4) of a human IgG2 constant region containing two inserted mutations (V234A and G237A) was added to the C terminus thereof (SEQ ID NO: 5). Next, a signal sequence (SEQ ID NO: 6) was added to the N terminus of the amino acid sequence (SEQ ID NO: 2) of the light chain variable region (L4) of the humanized anti-human fractalkine antibody (H3-2L4), and the amino acid sequence (SEQ ID NO: 7) of a human IgK constant region was added to the C terminus thereof (SEQ ID NO: 8). These amino acid sequences (SEQ ID NOs: 5 and 8) were converted to gene sequences optimal for expression in CHO cells. Respective sequences in which a recognition sequence for restriction enzyme HindIII and a Kozak sequence were added to the 5′ ends of the gene sequences and a stop codon and a recognition sequence for restriction enzyme EcoRI were added to the 3′ ends thereof were totally synthesized (SEQ ID NOs: 9 and 10).

The sequences defined by SEQ ID NOs: 1 to 10 are each as follows:

Amino acid sequence of heavy chain variable region
(H3-2)
(SEQ ID NO: 1)
QVQLVQSGAEVKKPGASVKVSCKASGYTFTNYYIHWVKQAPGQGLEWIGW
IYPGDGSPKFNERFKGRTTLTADKSTNTAYMLLSSLRSEDTAVYFCATGP
TDGDYFDYWGQGTTVTVSS
Amino acid sequence of light chain variable region
(L4)
(SEQ ID NO: 2)
DIQMTQSPSSLSASVGDRVTITCRASGNIHNFLAWYQQKPGKAPKLLIYN
EKTLADGVPSRFSGSGSGTDYTLTISSLQPEDFATYFCQQFWSTPYTFGG
GTKVEIK
Signal sequence
(SEQ ID NO: 3)
MEWSWVFLFFLSVTIGVHS
Amino acid sequence of human IgG2 constant region
containing two inserted mutations (V234A and
G237A)
(SEQ ID NO: 4)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPAAAPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
(SEQ ID NO: 5)
MEWSWVFLFFLSVTTGVHSQVQLVQSGAEVKKPGASVKVSCKASGYTFTN
YYIHWVKQAPGQGLEWIGWIYPGDGSPKFNERFKGRTTLTADKSTNTAYM
LLSSLRSEDTAVYFCATGPTDGDYFDYWGQGTTVTVSSASTKGPSVFPLA
PCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAP
PAAAPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIE
KTISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEALH
NHYTQKSLSLSPGK
Signal sequence
(SEQ ID NO: 6)
MSVPTQVLGLLLLWLTDARC
Amino acid sequence of human Igκ constant region
(SEQ ID NO: 7)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
(SEQ ID NO: 8)
MSVPTQVLGLLLLWLTDARCDIQMTQSPSSLSASVGDRVTITCRASGNIH
NFLAWYQQKPGKAPKLLIYNEKTLADGVPSRFSGSGSGTDYTLTISSLQP
EDFATYFCQQFWSTPYTFGGGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC
(SEQ ID NO: 9)
AAGCTTGCCGCCACCATGGAATGGTCCTGGGTGTTCCTGTTCTTCCTGTC
CGTGACCACCGGCGTGCACTCCCAGGTGCAGCTGGTGCAGTCTGGCGCCG
AAGTGAAGAAACCTGGCGCCTCCGTGAAGGTGTCCTGCAAGGCCTCCGGC
TACACCTTCACCAACTACTACATCCACTGGGTGAAACAGGCCCCAGGACA
GGGCCTGGAATGGATCGGCTGGATCTACCCCGGCGACGGCTCCCCCAAGT
TCAACGAGCGGTTCAAGGGCCGGACCACCCTGACCGCCGACAAGTCCACC
AACACCGCCTACATGCTGCTGTCCTCCCTGCGGAGCGAGGATACCGCCGT
GTACTTCTGCGCCACCGGCCCTACCGACGGCGACTACTTCGACTACTGGG
GCCAGGGCACCACCGTGACCGTGTCCTCTGCCTCCACCAAGGGCCCCTCC
GTGTTCCCTCTGGCCCCTTGCTCCCGGTCCACCTCTGAGTCTACCGCCGC
TCTGGGCTGCCTGGTCAAAGACTACTTCCCCGAGCCTGTGACAGTGTCCT
GGAACTCTGGCGCCCTGACCTCTGGAGTGCATACCTTCCCTGCCGTGCTG
CAGTCATCCGGCCTGTACTCCCTGTCCTCCGTGGTGACAGTGCCCTCCTC
CAACTTCGGCACCCAGACCTACACCTGTAACGTGGACCACAAGCCCTCCA
ACACCAAGGTGGACAAGACCGTGGAACGGAAGTGCTGCGTGGAATGCCCC
CCCTGTCCTGCCCCTCCTGCCGCCGCTCCTTCCGTGTTTCTGTTCCCCCC
AAAGCCCAAGGACACCCTGATGATCTCCCGGACCCCCGAAGTGACCTGCG
TGGTGGTGGACGTGTCCCACGAGGACCCCGAGGTGCAGTTCAATTGGTAC
GTGGACGGCGTGGAAGTGCACAACGCCAAGACCAAGCCCAGAGAGGAACA
GTTCAACTCCACCTTCCGGGTGGTGTCCGTGCTGACCGTGGTGCACCAGG
ACTGGCTGAACGGCAAAGAGTACAAGTGCAAGGTCTCCAACAAGGGCCTG
CCTGCCCCCATCGAAAAGACCATCAGCAAGACCAAGGGCCAGCCCCGCGA
GCCCCAGGTGTACACACTGCCCCCCAGCCGGGAAGAGATGACCAAGAACC
AGGTGTCCCTGACCTGTCTGGTGAAAGGCTTCTACCCCTCCGATATCGCC
GTGGAATGGGAGTCCAACGGACAGCCCGAGAACAACTACAAGACCACCCC
CCCCATGCTGGACTCCGACGGCTCATTCTTCCTGTACTCCAAGCTGACAG
TGGACAAGTCCCGGTGGCAGCAGGGCAACGTGTTCTCCTGCAGCGTGATG
CACGAGGCCCTGCACAACCACTACACCCAGAAGTCCCTGAGCCTGAGCCC
CGGCAAGTGATGAATTC
(SEQ ID NO: 10)
AAGCTTGCCGCCACCATGTCCGTGCCCACCCAGGTGCTGGGCCTGCTGCT
GCTGTGGCTGACCGACGCCAGATGCGACATCCAGATGACCCAGTCCCCCT
CCAGCCTGTCCGCCTCTGTGGGCGACAGAGTGACCATCACCTGTCGGGCC
TCCGGCAACATCCACAACTTTCTGGCCTGGTATCAGCAGAAGCCCGGCAA
GGCCCCCAAGCTGCTGATCTACAACGAAAAGACCCTGGCCGACGGCGTGC
CCTCCAGATTCTCCGGCTCTGGCTCCGGCACCGACTACACCCTGACCATC
TCCAGCCTGCAGCCCGAGGACTTCGCCACCTACTTTTGCCAGCAGTTCTG
GTCCACCCCCTACACCTTCGGCGGAGGCACCAAGGTGGAAATCAAGCGGA
CCGTGGCCGCTCCCTCCGTGTTCATCTTCCCACCCTCCGACGAGCAGCTG
AAGTCCGGCACCGCCTCCGTGGTGTGCCTGCTGAACAACTTCTACCCCCG
CGAGGCCAAGGTGCAGTGGAAGGTGGACAACGCCCTGCAGTCCGGCAACT
CCCAGGAATCCGTCACCGAGCAGGACTCCAAGGACAGCACCTACTCCCTG
TCCTCCACCCTGACCCTGTCCAAGGCCGACTACGAGAAGCACAAGGTGTA
CGCCTGCGAAGTGACCCACCAGGGCCTGTCCAGCCCCGTGACCAAGTCCT
TCAACCGGGGCGAGTGCTGATGAATTC

The totally synthesized gene sequence encoding the heavy chain was cleaved with restriction enzymes HindIII and EcoRI and inserted to the HindIII-EcoRI site of a pEE6.4 vector (Lonza Group Ltd.). The totally synthesized gene sequence encoding the light chain was cleaved with restriction enzymes HindIII and EcoRI and inserted to the HindIII-EcoRI site of a pEE12.4 vector (Lonza Group Ltd.). Each vector was cleaved with restriction enzymes NotI and PvuI. The heavy chain and light chain vector fragments were ligated to construct an expression vector.

### 1-2. Construction of Cell Line Expressing Antibody H3-2L4 and Obtainment of Antibody H3-2L4

CHOK1SV cells (Lonza Group Ltd.) acclimatized to a medium of CD-CHO/6 mM L-glutamine were transfected with the prepared expression vector by electroporation. The cells thus transfected were selected in a 37° C./10% CO₂ environment in a medium of CD-CHO/50 μM MSX to obtain cells expressing the antibody of interest. Then, the cells were cloned to prepare a cell bank. The prepared cell bank was revived and cultured, and the culture supernatant was purified by chromatography to obtain the antibody H3-2L4 of interest.

The antibody H3-2L4 thus obtained had a heavy chain and a light chain having the amino acid sequences represented by SEQ ID NOs: 11 and 12, respectively.

Full-length heavy chain of H3-2L4
(SEQ ID NO: 11)
QVQLVQSGAEVKKPGASVKVSCKASGYTFTNYYIHWVKQAPGQGLEWIGW
IYPGDGSPKFNERFKGRTTLTADKSTNTAYMLLSSLRSEDTAVYFCATGP
TDGDYFDYWGQGTTVTVSSASTKGPSVFPLAPCSRSTSESTAALGCLVKD
YFPEPVTVSWNSGALTSGVHTFPAVLQSSGLYSLSSVVTVPSSNFGTQTY
TCNVDHKPSNTKVDKTVERKCCVECPPCPAPPAAAPSVFLFPPKPKDTLM
ISRTPEVTCVVVDVSHEDPEVQFNWYVDGVEVHNAKTKPREEQFNSTFRV
VSVLTVVHQDWLNGKEYKCKVSNKGLPAPIEKTISKTKGQPREPQVYTLP
PSREEMTKNQVSLTCLVKGFYPSDIAVEWESNGQPENNYKTTPPMLDSDG
SFFLYSKLTVDKSRWQQGNVFSCSVMHEALHNHYTQKSLSLSPGK
Full-length light chain of H3-2L4
(SEQ ID NO: 12)
DIQMTQSPSSLSASVGDRVTITCRASGNIHNFLAWYQQKPGKAPKLLIYN
EKTLADGVPSRFSGSGSGTDYTLTISSLQPEDFATYFCQQFWSTPYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The amino acid sequences of the heavy chain variable region and the light chain variable region of the antibody H3-2L4 were each as follows:

Heavy chain variable region of H3-2L4
(SEQ ID NO: 13)
QVQLVQSGAEVKKPGASVKVSCKASGYTFTNYYIHWVKQAPGQGLEWIGW
IYPGDGSPKFNERFKGRTTLTADKSTNTAYMLLSSLRSEDTAVYFCATGP
TDGDYFDYWGQGTTVTVSS
Light chain variable region of H3-2L4
(SEQ ID NO: 14)
DIQMTQSPSSLSASVGDRVTITCRASGNIHNFLAWYQQKPGKAPKLLIYN
EKTLADGVPSRFSGSGSGTDYTLTISSLQPEDFATYFCQQFWSTPYTFGG
GTKVEIK

The amino acid sequences of CDR-H1 to CDR-H3 and CDR-L1 to CDR-L3 of the antibody H3-2L4 were as follows:

CDR-H1 of H3-2L4
(SEQ ID NO: 15)
NYYIH
CDR-H2 of H3-2L4
(SEQ ID NO: 16)
WIYPGDGSPKFNERFKG
CDR-H3 of H3-2L4
(SEQ ID NO: 17)
GPTDGDYFDY
CDR-L1 of H3-2L4
(SEQ ID NO: 18)
RASGNIHNFLA
CDR-L2 of H3-2L4
(SEQ ID NO: 19)
NEKTLAD
CDR-L3 of H3-2L4
(SEQ ID NO: 20)
QQFWSTPYT

### Example 2

### Single-Administration Test

A clinical phase 1 trial targeting healthy adult males was conducted for evaluating safety, tolerability, and pharmacokinetics by a single subcutaneous administration of H3-2L4.

### 2-1. [Clinical Trial Design]

This clinical trial was a monocentric, randomized, double-blind, placebo-controlled single-ascending dose study targeting healthy Japanese adult males and having a primary object to evaluate safety and tolerability by a single subcutaneous administration of H3-2L4. In this clinical trial, 32 subjects were divided into 4 cohorts (50, 100, 200, and 400 mg groups). H3-2L4 was subcutaneously administered by a single administration to 6 out of the 8 subjects in each cohort, and a placebo was subcutaneously administered by a single administration to the remaining 2 subjects.

This clinical trial was composed of a screening period, an observation hospitalization period, an administration hospitalization period, and a follow-up period.

The screening test was carried out within 28 days to 2 days before administration of a study drug, and the test for the observation hospitalization period was carried out on the day before administration to confirm eligibility. The subjects confirmed to be eligible were randomly allocated to a H3-2L4 or placebo group on the basis of an allocation list prepared by a person in charge of allocation. The dosing interval of each of the subjects was set to 30 minutes or longer. This clinical trial was a single-administration test, and the administration to each subject was performed only once independently of mealtime.

The subjects were observed and investigated by hospitalization for 2 weeks after the administration and then observed and investigated as outpatients until 8 weeks after the completion of the administration.

Cohort progression was conducted in the judgment of a principal investigator, and after a lapse of 7 or more days after the completion of administration to the final subject in a previous cohort, the administration to a next cohort was started.

The types of the study drug used in this trial and the formulations of the study drugs were as described below.

TABLE 1
Types of study drugs

| Type | Dosage form and content | Manufacturer |
|---|---|---|
| H3-2L4 | Aqueous solution containing 100 | Eisai Co., |
| | mg of H3-2L4 in one vial (1 mL) | Ltd. |
| Placebo | Aqueous solution free from H3- | |
| | 2L4 in one vial (1 mL) | |

TABLE 2
Formulation

| Component | Concentration | |
|---|---|---|
| H3-2L4 | 100 | mg/mL |
| Phosphate Buffer (pH 5.8) | 25 | mM |
| Sucrose | 200 | mM |
| Glycine | 50 | mM |

| | |
|---|---|
| Polysorbate 80 | 0.05% |

### 2-2. [Administration of Study Drug]

H3-2L4 or a placebo was subcutaneously administered to the brachial region or the abdomen according to Table 3 below. For the first cohort (50 mg group) and the second cohort (100 mg group), 0.5 mL and 1.0 mL, respectively, were subcutaneously administered to either of the right or left upper arm. For the third cohort (200 mg group), 1.0 mL (2.0 mL in total) was subcutaneously administered to each of the right and left upper arms. For the fourth cohort (400 mg group), 1.0 mL (4.0 mL in total) was subcutaneously administered to each of a total of 4 regions, the right and left upper arms and the right and left flanks.

TABLE 3
Method for administering study drug

| Cohort | Study drug | Administration method |
|---|---|---|
| 1 | H3-2L4 50 mg or | Subcutaneous administration |
| | placebo | of 0.5 mL to either of the |
| | | right or left upper arm |
| 2 | H3-2L4 100 mg or | Subcutaneous administration |
| | placebo | of 1.0 mL to either of the |
| | | right or left upper arm |
| 3 | H3-2L4 200 mg or | Subcutaneous administration |
| | placebo | of 1.0 mL (2.0 mL in total) |
| | | to each of the right and |
| | | upper arms |
| 4 | H3-2L4 400 mg or | Subcutaneous administration |
| | placebo | of 1.0 mL (4.0 mL in total) |
| | | to each of a total of 4 |
| | | regions, the right and left |
| | | upper arms and the right |
| | | and left flanks |

### 2-3. [Analysis of Serum H3-2L4 Concentration]

The serum H3-2L4 concentration was measured by use of a validated measurement method. Specifically, serum H3-2L4 was bound to human fractalkine immobilized on a microplate and reacted with a ruthenium-labeled anti-H3-2L4 rabbit polyclonal antibody. Then, the electrochemical luminescence intensity was measured using Sector Imager 6000 (Meso Scale Discovery) for quantification. Blood collection for the serum H3-2L4 concentration measurement was carried out at the time of blood collection shown in Table 4 below.

TABLE 4
Time of blood collection for pharmacokinetic evaluation

| | | | | Tolerance of | |
|---|---|---|---|---|---|
| | | | | time of | |
| | Time of blood collection (time | | | blood | |
| Period | elapsed from administration at day 1) | | | collection | |
| Day 1 | Immediately before administration | | | −3 | hr |
| | 6 hr | | | ±5 | min |
| | 12 hr | | | | |
| Day 2 | 24 hr | The same hour as that of | | ±1 | hr |
| Day 3 | 48 hr | administration at day 1 | | | |
| Day 4 | 72 hr | | | | |
| Day 5 | 96 hr | | | | |
| Day 6 | 120 hr | | | | |
| Day 7 | 144 hr | | | | |
| Day 8 | 168 hr | | | | |
| Day 9 | 192 hr | | | | |
| Day 11 | 240 hr | | | | |
| Day 13 | 288 hr | | | | |
| Day 15 | 336 hr | | | | |
| Day 22 | 504 hr | 3 weeks | On the same | ±1 | d |
| | | later | day of week as | | |
| Day 29 | 672 hr | 4 weeks | that of | | |
| | | later | administration | | |
| Day 36 | 840 hr | 5 weeks | at day 1 | | |
| | | later | | | |
| Day 43 | 1008 hr | 6 weeks | | | |
| | | later | | | |
| Day 50 | 1176 hr | 7 weeks | | | |
| | | later | | | |
| Day 57 | 1344 hr | 8 weeks | | | |
| | | later | | | |

### 2-4. [Pharmacokinetic Analysis]

The pharmacokinetic analysis was carried out on the basis of the serum H3-2L4 concentration data using a set of subjects having data that permitted calculation of one or more pharmacokinetic parameters (hereinafter, referred to as a pharmacokinetic analysis set). The summary statistic of the serum H3-2L4 concentration was calculated for each specified time with respect to each dose. A diagram showing change in the serum H3-2L4 concentration was prepared, and pharmacokinetic parameters including Cₘₐₓ, tₘₐₓ, AUC₍₀₋ₜ₎, AUC_{(0-inf)}, t_{1/2}, CL/F, and V_{z}/F were calculated by non-compartmental analysis using the serum H3-2L4 concentration. The analysis was carried out using SAS, WinNonlin, Pharsight Knowledgebase Server, Microsoft Excel, and S-PLUS.

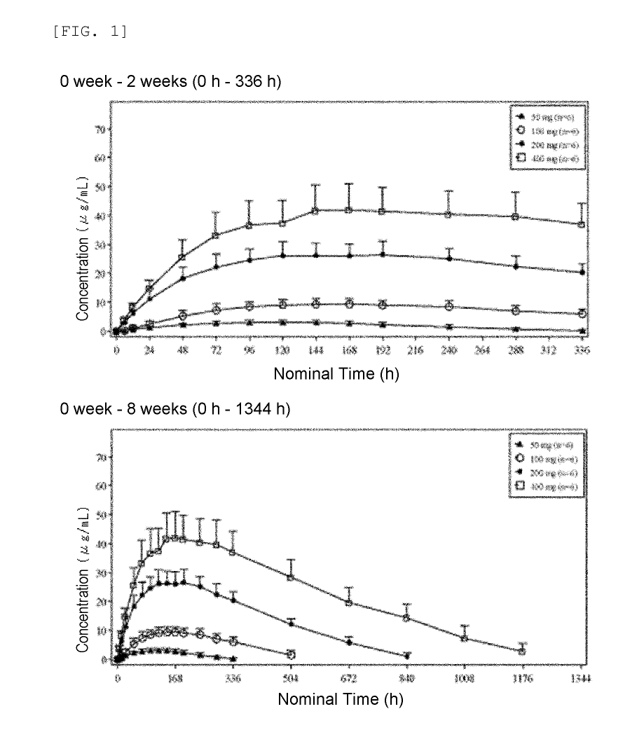

The results thus calculated are shown in Table 5 below and FIG. 1. In the table, each PK parameter other than tₘₐₓ is indicated by an arithmetic mean value, and the numerical value within the parentheses represents standard deviation. tₘₐₓ is indicated by a median value, and the numerical values within the parenthesis represent the smallest and largest values. In the table, a represents n=3.

TABLE 5
Summary of pharmacokinetic parameter when H3-2L4 was subcutaneously
administered by single administration to healthy human adult

| | H3-2L4 | | | |
|---|---|---|---|---|
| | 50 mg | 100 mg | 200 mg | 400 mg |
| PK Parameter | (n = 6) | (n = 6) | (n = 6) | (n = 6) |
| Cₘₐₓ (μg/mL) | 3.14 (0.788) | 9.66 (1.88) | 27.3 (4.47) | 43.1 (8.88) |
| tₘₐₓ (h) | 120 (48-144) | 144 (120-240) | 156 (120-192) | 156 (72-288) |
| AUC₍₀₋ₜ₎ (μg · h/mL) | 602 (186) | 2990 (745) | 11900 (2170) | 26600 (6770) |
| AUC_{(0-336 h)} (μg · h/mL) | 662 (272) ^{a} | 2430 (514) | 7420 (1190) | 11800 (2300) |
| t_{1/2, β} (h) | — | — | 242 (30.5) | 349 (37.2) |
| t_{1/2, γ} (h) | 40.8 (8.42) | 70.6 (19.5) | — | — |
| DN Cₘₐₓ | 0.0629 (0.0158) | 0.0966 (0.0188) | 0.137 (0.0222) | 0.108 (0.0221) |
| ([μg/mL]/[mg]) | | | | |
| DN AUC₍₀₋ₜ₎ | 12.0 (3.72) | 29.9 (7.45) | 59.5 (10.8) | 66.6 (16.9) |
| ([(μg · h/mL)]/[mg]) | | | | |

As a result of the analysis, the pharmacokinetics when H3-2L4 was subcutaneously administered by a single administration was considered to exhibit a 3-phase profile having an absorption phase and two elimination phases (β phase and γ phase). After the single subcutaneous administration, H3-2L4 was slowly absorbed and reached Cₘₐₓ in 120 to 156 hours (median value). After the antibody reached Cₘₐₓ, a gradual elimination phase (β phase) was observed for the high concentrations, while a steeper final elimination phase (γ phase) was observed for the low concentrations. The boundary point between the γ phase and the β phase was considered to be around 10 μg/mL. The 50 and 100 mg groups exhibited a 2-phase profile, suggesting that the γ phase appeared subsequently to Cₘₐₓ. Since the 200 and 400 mg groups fell short of the number of blood samples necessary for accurately evaluating the γ phase as the final elimination phase, the pharmacokinetic parameters dependent on the γ phase were not able to be calculated. t_{1/2,β} was longer than t_{1/2,γ} and was prolonged with increase in dose. H3-2L4 exhibited nonlinear pharmacokinetics in the dose range of 50 to 400 mg.

### Example 3

### Multiple-Administration Test

A clinical phase ½ trial targeting rheumatoid arthritis patients was conducted for evaluating safety, tolerability, pharmacokinetics, and effectiveness for rheumatoid arthritis by repeated subcutaneous administrations of H3-2L4.

### 3-1. [Clinical Trial Design]

This clinical trial was a multicenter, open-label, uncontrolled, multiple-ascending dose (MAD) study targeting Japanese patients with rheumatoid arthritis and having a primary object to evaluate safety and tolerability by repeated subcutaneous administrations of H3-2L4 for 12 weeks. Randomization was not carried out. In this clinical trial, a 100 mg administration group and a 200 mg administration group involved 12 subjects and 15 subjects, respectively, and after confirmation of the safety of the 100 mg administration group, the test progressed to the 200 mg administration group. The safety evaluation in an administration period of all of the subjects in the 200 mg administration group was further completed to confirm the absence of problems with safety. Then, the test was carried out targeting 10 subjects in a 400 mg administration group.

This clinical trial was composed of a screening period, an observation period, an administration period, a continuous administration period, and a follow-up period.

The screening test was carried out within 42 days to 2 days before administration of a study drug, and the test for the observation period was carried out on the day before initial administration of the study drug or before the administration on this day. H3-2L4 was administered to the subjects confirmed to be eligible.

Selection criteria for the eligibility were as follows:
(1) those at age 20 years or older and younger than 65 years;
(2) rheumatoid arthritis (RA) patients who satisfied the ACR Classification Criteria or the 2010 ACR/EULAR Classification Criteria;
(3) patients who received either of the following treatments, or both, before the start of screening and were found to have 4 or more tender joints (in 68 joints) and 4 or more swollen joints (in 66 joints) by evaluation in the screening period and the observation period:

patients who received 3-month or longer treatment with methotrexate (MTX). However, patients who had to discontinue the administration due to adverse reaction were not limited by the history of treatment for 3 months or longer, and

patients who received 3-month or longer treatment with an anti-TNF formulation. However, the anti-TNF formulation used in the treatment was limited to any one of adalimumab, infliximab, golimumab, certolizumab pegol, and etanercept (the anti-TNF formulation includes a biosimilar);
(4) patients who had not received any biological product (tocilizumab, abatacept, etc.) other than the anti-TNF formulation or two or more anti-TNF formulations in the past;
(5) patients who exhibited 0.6 mg/dL or higher of high sensitive CRP (hs-CRP) or an erythrocyte sedimentation rate (ESR) of 28 mm/hr or higher in the screening period; and
(6) patients who had a body weight of 30 kg or higher and 100 kg or lower at the time of screening.

In the administration period, H3-2L4 was administered a total of 7 times (week 0, week 1, week 2, and subsequently, every two weeks until week 10). The same dose was further administered 20 times every two weeks (40 weeks) to subjects who had no problem with safety in evaluation 2 weeks after the completion of the 7th administration (at week 12), exhibited 20% or more improvement in both of the tender joint count and the swollen joint count from the observation period, and desired continuous administration (continuous administration period). As for the continuous administration period of the 400 mg administration group, the dose was able to be decreased to 200 mg, which was also able to be brought back to 400 mg thereafter, in the judgment of a principal investigator or a subinvestigator.

The type of the study drug used in this trial and the formulation of the therapeutic agent were as follows.

TABLE 6
Study drug

| Type | Dosage form and content | Manufacturer |
|---|---|---|
| H3-2L4 | Aqueous solution containing 100 | Eisai Co., |
| | mg of H3-2L4 in one vial (1 mL) | Ltd. |

TABLE 7
Formulation

| Component | Concentration | |
|---|---|---|
| H3-2L4 | 100 | mg/mL |
| Phosphate Buffer (pH 5.8) | 25 | mM |
| Sucrose | 200 | mM |
| Glycine | 50 | mM |

| | |
|---|---|
| Polysorbate 80 | 0.05% |

### 3-2. [Administration of Study Drug]

H3-2L4 was subcutaneously administered to the upper arms, the abdomen, or the femoral regions according to Table 8 below. For the 100 mg administration group, 1.0 mL was subcutaneously administered to any one of the right and left upper arms, the right and left flanks, and the right and left femoral regions. For the 200 mg administration group, 1.0 mL (2.0 mL in total) was subcutaneously administered to each of any two of the right and left upper arms, the right and left flanks, and the right and left femoral regions. For the 400 mg administration group, 1.0 mL (4.0 mL in total) was subcutaneously administered to each of any four of the right and left upper arms, the right and left flanks, and the right and left femoral regions. As for the 400 mg administration group, 2.0 mL (4.0 mL in total) was able to be subcutaneously administered to each of any two thereof as long as a principal investigator or a subinvestigator judged that this subcutaneous administration was properly achieved.

TABLE 8
Dose and administration method of H3-2L4

| Administration | Study | |
|---|---|---|
| group | drug | Administration method |
| 100 mg | H3-2L4 | Subcutaneous administration of |
| administration | 100 mg | 1.0 mL to any one of the right |
| group | | and left upper arms, the right |
| | | and left flanks, and the right |
| | | and left femoral regions |
| 200 mg | H3-2L4 | Subcutaneous administration of |
| administration | 200 mg | 1.0 mL (2.0 mL in total) to |
| group | | each of any two of the right |
| | | and left upper arms, the right |
| | | and left flanks, and the right |
| | | and left femoral regions |
| 400 mg | H3-2L4 | Subcutaneous administration of |
| administration | 400 mg | 1.0 mL (4.0 mL in total) to |
| group | | each of any four of the right |
| | | and left upper arms, the right |
| | | and left flanks, and the right |
| | | and left femoral regions |
| | | (subcutaneous administration |
| | | of 2.0 mL (4.0 mL in total) to |
| | | each of any two thereof was |
| | | also possible as long as a |
| | | principal investigator or a |
| | | subinvestigator judged that |
| | | this subcutaneous |
| | | administration was properly |
| | | achieved) |

The administration of the study drug was carried out after the completion of all investigations (except for findings about administration sites) scheduled on that day.

The same dose was further administered 20 times every two weeks (40 weeks) to subjects who had no problem with safety in evaluation 2 weeks after the completion of the 7th administration (at week 12), exhibited 20% or more improvement in both of the tender joint count and the swollen joint count from the observation period, and desired continuous administration. The subjects who progressed to the continuous administration period were observed and investigated until 52 weeks after the initial administration. The administration of the study drug was carried out after the completion of all investigations (except for findings about administration sites) scheduled on that day. The dosing intervals of the study drug were set to 7 days or longer (6-day or longer resting period). If a dosing interval was shorter than 7 days because of changing the day of observation and investigation, only the observation and investigation were carried out with a cessation of the study drug.

### 3-3. [Pharmacokinetics and Drug Potency]

The serum H3-2L4 concentration and the serum total FKN concentration were measured by use of validated measurement methods. The serum H3-2L4 concentration was measured by the method described in 2-4 of Example 2. The serum total FKN concentration was measured by sandwich ELISA using two types of anti-human fractalkine monoclonal antibodies 1F3 and 3A5-2 described in WO2011/052799. Specifically, serum total FKN was captured onto 1F3 immobilized on a plate, then sandwiched with horseradish peroxidase (HRP)-labeled 3A5-2, and further reacted with an HRP substrate 3,3′,5,5′-tetramethylbenzidine (TMB). The luminescence value was measured using an auto-reader (Microplate reader Envision, PerkinElmer Co., Ltd.) to determine the serum total FKN concentration.

The pharmacokinetic analysis was carried out on the basis of the serum H3-2L4 concentration data using a set of subjects who received the study drug and had one or more evaluable serum EH3-2L4 concentration data points (hereinafter, referred to as a pharmacokinetic analysis set). The summary statistic of the serum H3-2L4 concentration was calculated for each specified time with respect to each dose. A diagram showing change in the serum H3-2L4 concentration was prepared.

### 3-3-1. [Serum H3-2L4 Concentration]

Blood collection for the serum H3-2L4 concentration measurement was carried out at the times (counted, starting at the administration start day (day 1 (week 0)) (the same holds true for the description below); before administration (at day 1) and at days 8, 15±1, 29±3, 43±3, 57±3, 71±3, and 85±3) shown in the implementation schedule. As a rule, the blood collection for the serum H3-2L4 concentration measurement was carried out at any one point after 5 days (±3 days) from the administration at week 2, week 4, or week 6. For the subjects who progressed to the continuous administration period, the blood collection for the serum H3-2L4 concentration measurement was carried out according to the implementation schedule, as in the administration period.

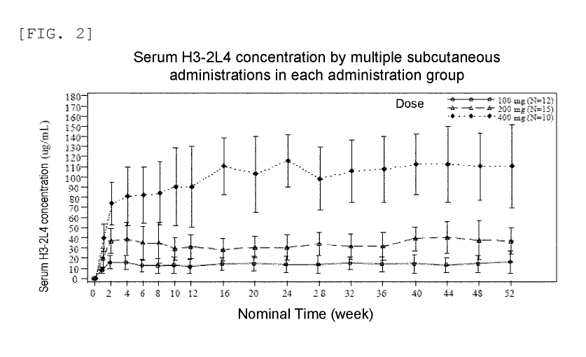

The serum H3-2L4 concentrations (indicated by arithmetic mean values) of the 100 mg administration group, the 200 mg administration group, and the 400 mg administration group over the administration period (12 weeks) and the continuous administration period (40 weeks) are shown in FIG. 2.

### 3-3-2. [Serum Total FKN Concentration]

Blood collection for the serum total FKN concentration measurement was carried out at the times of blood collection (before administration (at day 1) and at days 8, 15±1, 29±3, 43±3, 57±3, 71±3, and 85±3) shown in the implementation schedule. For the subjects who progressed to the continuous administration period, the blood collection for the serum total FKN concentration measurement was carried out according to the implementation schedule, as in the administration period.

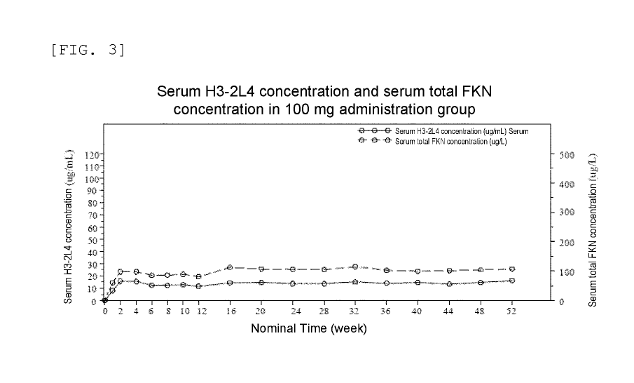

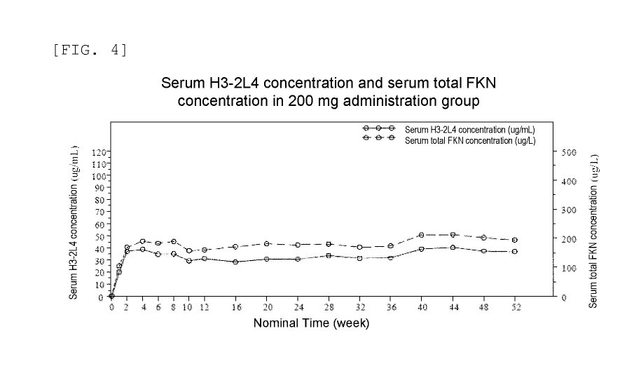

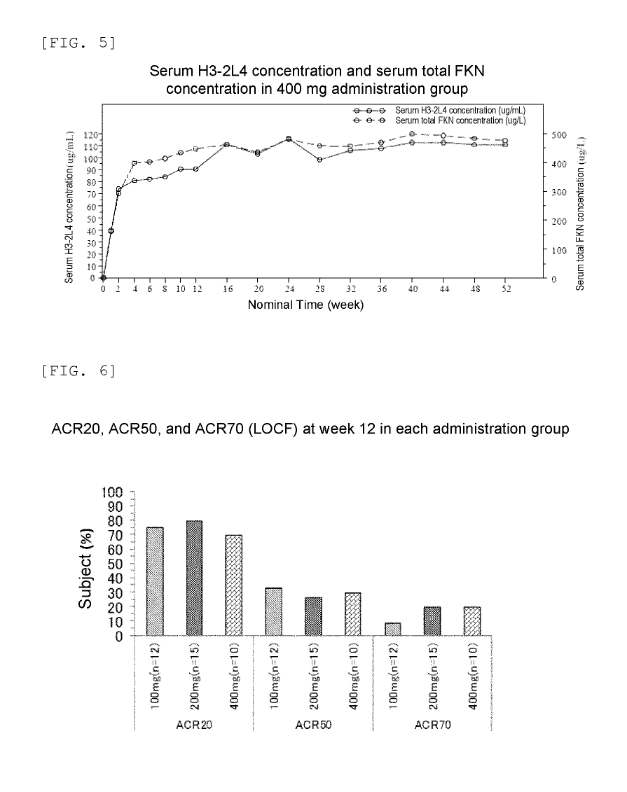

The summary statistics (indicated by arithmetic mean values) of the serum total FKN concentrations of the 100 mg administration group and the 200 mg administration group over the administration period (12 weeks) and the continuous administration period (40 weeks) are shown in FIGS. 3 and 4, respectively, together with the summary statistics of the serum H3-2L4 concentrations of these administration groups. Also, the summary statistics (indicated by arithmetic mean values) of the serum total FKN concentrations of the 400 mg administration group over the administration period and the continuous administration period are shown in FIG. 5 together with the summary statistics of the serum H3-2L4 concentrations.

### 3-4. [Effectiveness]

The effectiveness was analyzed using a set of subjects who received the study drug and had one or more evaluable post-administration effectiveness data points of the study drug (full analysis set (FAS)).

The effectiveness of the study drug for the rheumatoid arthritis was evaluated by calculating ACR20, ACR50, and ACR70 response rates and two-sided confidence intervals of 95% thereof for each time of evaluation with respect to each dose. The summary statistics of ACR evaluation items, i.e., tender and swollen joint counts, evaluation using VAS (patient assessment of pain, patient global assessment of disease activity, and provider global assessment of disease activity), patients' health assessment questionnaire (HAQ), and hs-CRP and ESR values and their amounts of change and rates of change were calculated for each time of evaluation. Likewise, the summary statistics of DAS28-ESR, DAS28-CRP, SDAI (simple disease activity index), and CDAI (clinical disease activity index) values and their amounts of change were calculated for each time of evaluation with respect to each dose. In addition, the respective remission rates of remission criteria (DAS28-ESR, DAS28-CRP, SDAI, CDAI, and Boolean) and two-sided confidence intervals of 95% thereof were calculated.

The analysis was carried out using SAS.

### 3-4-1. [ACR20, ACR50, and ACR70]

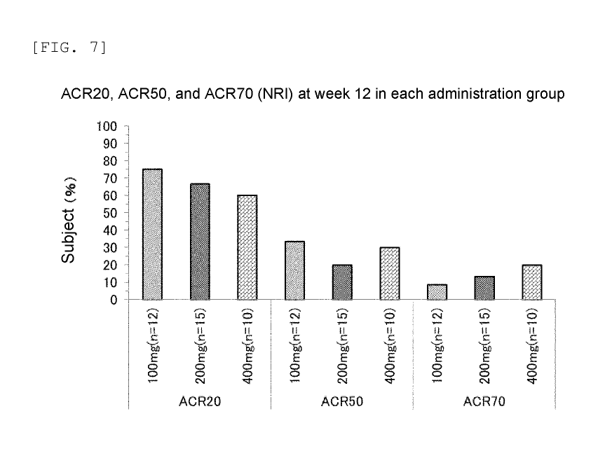

The results about ACR20, ACR50, and ACR70 at week 12 in each administration group are shown in FIGS. 6 (LOCF (last observation carried forward)) and 7 (NRI (non-responder imputation)). The proportions of subjects who achieved ACR20, ACR50, and ACR70 (LOCF) at week 12 were 75.0%, 33.3%, and 8.3%, respectively, in the 100 mg administration group, 80.0%, 26.7%, and 20.0%, respectively, in the 200 mg administration group, and 70.0%, 30.0%, and 20.0%, respectively, in the 400 mg administration group. Also, the proportions of subjects who achieved ACR20, ACR50, and ACR70 (NRI) at week 12 were 75.0%, 33.3%, and 8.3%, respectively, in the 100 mg administration group, 66.7%, 20.0%, and 13.3%, respectively, in the 200 mg administration group, and 60.0%, 30.0%, and 20.0%, respectively, in the 400 mg administration group.

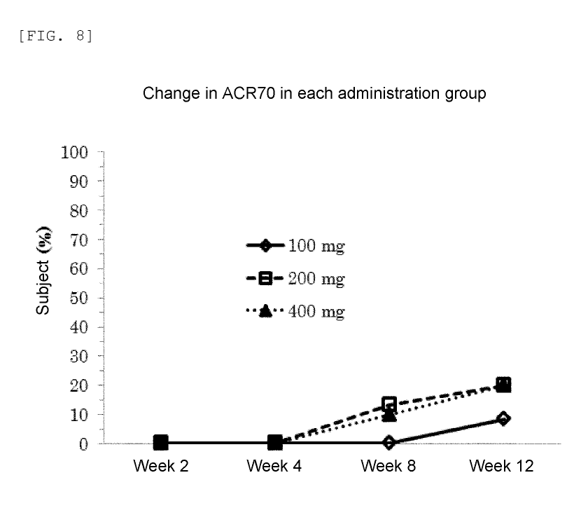

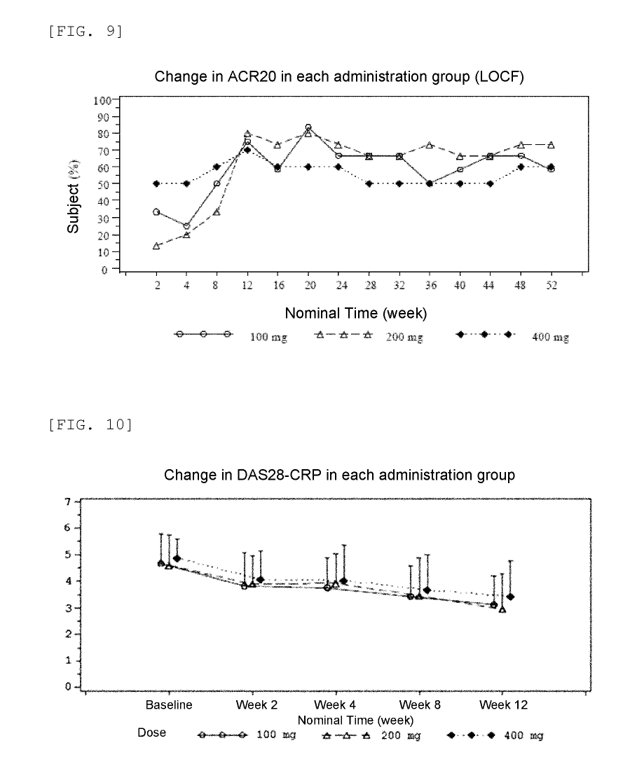

Change in the proportion of subjects who achieved ACR70 (LOCF) until week 12 in each administration group is shown in FIG. 8. As shown in FIG. 8, the 200 mg administration group and the 400 mg administration group exhibited amelioration effects earlier than the 100 mg administration group. As shown in FIG. 9, it was recognized that the subjects who progressed to the continuous administration period maintain the amelioration effects exhibited in the administration period.

### 3-4-2. [DAS28-CRP]

Change in DAS28-CRP (LOCF) in each administration group over the administration period (12 weeks) is shown in FIG. 10.

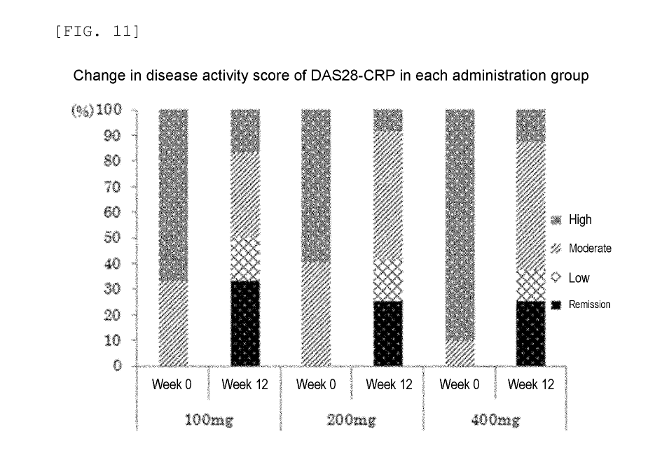

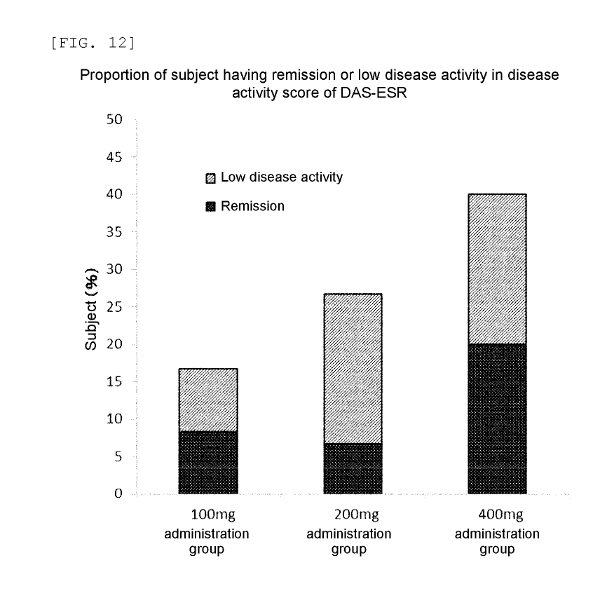

Change in the proportions of disease activity scores of DAS28-CRP in each administration group between before administration (at week 0) and at week 12 is shown in FIG. 11. As shown in FIG. 11, an improvement tendency was found in each administration group at 12 weeks after the start of administration. Remission was achieved in 20% or more of the subjects in each administration group. It was recognized that the subjects who progressed to the continuous administration period maintain the amelioration effects exhibited in the administration period.

### 3-4-3. [DAS28-ESR]

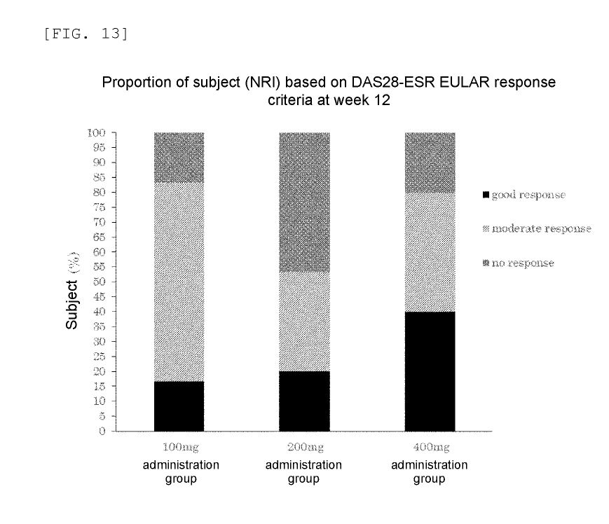

The proportion of subjects who reached remission or low disease activity in disease activity scores of DAS28-ESR (NRI) at week 12 in each administration group is shown in FIG. 12. The proportions of subjects who achieved remission or low disease activity are 16.7% (remission: 8.3%), 26.7% (remission: 6.7%), and 40.0% (remission: 20%), in the 100 mg administration group, the 200 mg administration group, and the 400 mg administration group, respectively. Also, the proportion of subjects based on EULAR response criteria of DAS28-ESR (NRI) at week 12 in each administration group is shown in FIG. 13. The subjects who achieved “good response” at week 12 accounted for 16.7%, 20%, and 40% of the 100 mg administration group, the 200 mg administration group, and the 400 mg administration group, respectively. These results demonstrated that amelioration effects are enhanced with increase in dose. It was recognized that the subjects who progressed to the continuous administration period maintain the amelioration effects exhibited in the administration period.

### 3-4-4. [SDAI and Boolean]

The SDAI remission rates at week 12 in the 100 mg administration group and the 200 mg administration group were 16.7% and 20.0%, respectively.

The Boolean remission rates at week 12 in the 100 mg administration group and the 200 mg administration group were 8.3% and 26.7%, respectively.

3-4-5. [Erythrocyte Sedimentation Rate (ESR) and High Sensitive CRP (hs-CRP)]

ESR was measured at the times (during the screening period, before administration (at day 1), and at days 15±1, 29±3, 57±3, and 85±3) shown in the implementation schedule, and at the time of discontinuation. ESR was further measured every 4 weeks until week 52 for the subjects who progressed to the continuous administration period. 1-hour ESR values were measured in the hospital, and the measurement values were described in case reports.

hs-CRP was measured at the times (during the screening period, before administration (at day 1), and at days 2, 9, 15±1, 29±3, 57±3, and 85±3) shown in the implementation schedule as a part of the biochemical examination of blood.

The serum H3-2L4 concentration at week 12, the area under the serum H3-2L4 concentration-time curve until week 12, and hs-CRP and ESR values at each time of evaluation (before administration (at week 0) and at week 12), and their rates of change, for each subject in the 100 mg administration group and the 200 mg administration group, are shown in Tables 9 and 10 below. The values in the tables were not based on analysis using SAS.

TABLE 9
Results about hs-CRP and ESR in 100 mg administration group

| | | Area under | | | | | | |
|---|---|---|---|---|---|---|---|---|
| | Lowest serum | lowest serum | Before | | | | Rate of change | |
| | H3-2L4 | H3-2L4 | administration | | | | at week 12 | |
| | concentration | concentration- | (week 0) | | Week 12 | | Rate of | Rate of |
| | at week 12 | time curve | hs-CRP | ESR | hs-CRP | ESR | change | change |
| Subject | (μg/ml) | (μg · h/mL) | (mg/dL) | (mm/h) | (mg/dL) | (mm/h) | in CRP | in ESR |
| A | | 58 | 2.94 | 74 | 3.26 | 64 | 0.11 | −0.14 |
| B | 19.9 | 1907 | 1.21 | 24 | 0.545 | 23 | −0.55 | −0.04 |
| C | 22.8 | 1657 | 0.355 | 41 | 0.508 | 33 | 0.43 | −0.20 |
| D | 8.89 | 1046 | 0.941 | 18 | 0.479 | 13 | −0.49 | −0.28 |
| E | 4.04 | 561 | 0.987 | 119 | 1.07 | 109 | 0.08 | −0.08 |
| F | 10.4 | 927 | 2.6 | 41 | 2.89 | 49 | 0.11 | 0.20 |
| G | 11 | 1072 | 0.317 | 50 | 0.143 | 26 | −0.55 | −0.48 |
| H | 5.56 | 632 | 0.197 | 38 | 0.324 | 40 | 0.64 | 0.05 |
| I | 10.6 | 1270 | 0.083 | 38 | 0.089 | 29 | 0.07 | −0.24 |
| J | 1.79 | 658 | 0.159 | 22 | 0.132 | 29 | −0.17 | 0.32 |
| K | 17.5 | 1379 | 0.268 | 67 | 0.075 | 22 | −0.72 | −0.67 |
| L | 16.6 | 1106 | 0.04 | 48 | 0.074 | 54 | 0.85 | 0.13 |

In the table, a rate of change in hs-CRP of −0.2 or less, or a rate of change in ESR of −0.2 or less is indicated in bold type. A lowest serum H3-2L4 concentration at week 12 equal to or lower than the measurement limit is indicated in blank.

### [Table 10]

Table 10 Results about hs-CRP and ESR in 200 mg administration group

In the table, a rate of change in CRP of −0.2 or less, or a rate of change in ESR of −0.2 or less is indicated in bold type. Test subjects EE, II, and LL discontinued the test.

When the results described above were stratified to subjects having a hs-CRP value of 0.6 mg/dL or higher (selection criterion value for eligibility in this trial) before administration (at week 0), a serum H3-2L4 concentration-dependent improvement tendency in the rate of change in hs-CRP was found in subjects having a lowest serum H3-2L4 concentration of approximately 10 μg/mL or higher at week 12. In fact, the arithmetic mean lowest serum H3-2L4 concentration at week 12 was 7.2 μg/mL for a group of the subjects who had a hs-CRP value of 0.6 mg/dL or higher before administration (at week 0) and a measurable lowest serum H3-2L4 concentration at week 12 and did not exhibit 20% or more improvement in hs-CRP in the 100 mg administration group, whereas this arithmetic mean value was 14.4 μg/mL for a group of the subjects who exhibited 20% or more improvement in hs-CRP. Likewise, the arithmetic mean lowest serum H3-2L4 concentration at week 12 was 24.2 μg/mL for a group of the subjects who exhibited 20% or more improvement in hs-CRP in the 200 mg administration group. In light of these results and the fact that the values reached the steady state at and after week 2, it was considered necessary that a pharmaceutical composition comprising H3-2L4 is administered so as to bring about a mean trough concentration of at least 10 μg/mL.

### 3-4-6. [Evaluation of Tender Joint Count and Swollen Joint Count]

The tender joint count and the swollen joint count were evaluated during the screening period, during the observation period, at 2 weeks, 4 weeks, 8 weeks, and 12 weeks after the start of the study drug administration, and at the time of discontinuation. These counts were further evaluated every 4 weeks until week 52 for the subjects who progressed to the continuous administration period. As a rule, the evaluation was carried out by the same physician-in-charge in each medical institute.

The tender joint count was evaluated by targeting 68 joints and applying pressure to or moving the joints. Also, the swollen joint count was evaluated in 66 joints obtained by excluding hip joints from the joints evaluated for the tender joint count.

The tender joint count and the swollen joint count at week 12 were decreased from the values before administration (at week 0), and the values of the decrease were 8.8±7.30 and 5.3±3.98, respectively, in the 100 mg administration group, 8.7±9.85 and 6.6±7.01, respectively, in the 200 mg administration group, and 10.4±11.27 and 8.8±8.30, respectively, in the 400 mg administration group. It was recognized that the subjects who progressed to the continuous administration period maintain the amelioration effects exhibited in the administration period.

### 3-4-7. [Evaluation Using Visual Analog Scale (VAS)]

The evaluation using VAS was carried out during the observation period, at 2 weeks, 4 weeks, 8 weeks, and 12 weeks after the start of the study drug administration, and at the time of discontinuation. The evaluation was further conducted every 4 weeks until week 52 for the subjects who progressed to the continuous administration period. As a rule, the evaluation was carried out by the same physician-in-charge in each medical institute. As in the other evaluations of effectiveness, it was recognized that the subjects who progressed to the continuous administration period maintain the amelioration effects exhibited in the administration period.

### 3-4-8. [Evaluation Using Health Assessment Questionnaire (HAQ)]

The evaluation using HAQ was carried out during the observation period, at 2 weeks, 4 weeks, 8 weeks, and 12 weeks after the start of the study drug administration, and at the time of discontinuation. The evaluation was further conducted every 4 weeks until week 52 for the subjects who progressed to the continuous administration period. As in the other evaluations of effectiveness, it was recognized that the subjects who progressed to the continuous administration period maintain the amelioration effects exhibited in the administration period.

1. A method for treating rheumatoid arthritis,
the method comprising subcutaneously administering at least 100 mg per dose of an anti-fractalkine antibody to a human in need thereof, wherein
the anti-fractalkine antibody is an antibody wherein:
a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDGS PKFNERFKGRTTLTADKSTNTAYMLLSSLRSEDTAVYFCATGPTDGDYFDYWGQGTTV TVSS);
a light chain variable region thereof comprises the amino acid sequence represented by SEQ ID NO: 14 (DIQMTQSPSSLSASVGDRVTITCRASGNIHNFLAWYQQKPGKAPKLLIYNEKTLADGVP SRFSGSGSGTDYTLTISSLQPEDFATYFCQQFWSTPYTFGGGTKVEIK);
the antibody comprises a constant region of human IgG2 isotype; and
a Fc region of the constant region of human IgG2 isotype contains V234A and G237A mutations.

2. The method according to claim 1, wherein
the method comprises subcutaneously administering 100 mg to 400 mg per dose of the anti-fractalkine antibody to a human.

3. The method according to claim 1, wherein
the method comprises subcutaneously administering 100 mg, 200 mg, or 400 mg per dose of the anti-fractalkine antibody to a human.

4. The method according to claim 1, wherein
the anti-fractalkine antibody is subcutaneously administered by a pharmaceutical composition which brings about the following:
when 100 mg of the anti-fractalkine antibody is subcutaneously administered by a single administration to a human,
mean Cₘₐₓ is a value that falls within the numerical range of 80% to 125% of 10 μg/mL, mean AUC_{(0-336 h)} is a value that falls within the numerical range of 80% to 125% of 2.4×10³ μg·h/mL, or
mean AUC₍₀₋ₜ₎ is a value that falls within the numerical range of 80% to 125% of 3.0×10³ μg·h/mL.

5. The method according to claim 1, wherein
the anti-fractalkine antibody is subcutaneously administered by a pharmaceutical composition which brings about the following:
when 200 mg of the anti-fractalkine antibody is subcutaneously administered by a single administration to a human,
mean Cₘₐₓ is a value that falls within the numerical range of 80% to 125% of 27 μg/mL,
mean AUC_{(0-336 h)} is a value that falls within the numerical range of 80% to 125% of 7.4×10³ μg·h/mL, or
mean AUC₍₀₋ₜ₎ is a value that falls within the numerical range of 80% to 125% of 1.2×10⁴ μg·h/mL.

6. The method according to claim 1, wherein
the anti-fractalkine antibody is subcutaneously administered by a pharmaceutical composition which brings about the following:
when 400 mg of the anti-fractalkine antibody is subcutaneously administered by a single administration to a human,
mean Cₘₐₓ is a value that falls within the numerical range of 80% to 125% of 43 μg/mL,
mean AUC_{(0-336 h)} is a value that falls within the numerical range of 80% to 125% of 1.2×10⁴ μg·h/mL, or
mean AUC₍₀₋ₜ₎ is a value that falls within the numerical range of 80% to 125% of 2.7×10⁴ μg·h/mL.

7. The method according to claim 1, wherein
the method comprises subcutaneously administering the anti-fractalkine antibody by multiple administrations at dosing intervals of once a week to once every two weeks.

8. The method according to claim 1, wherein
the anti-fractalkine antibody is subcutaneously administered such that the mean trough concentration thereof is 10 μg/mL or higher.

9. The method according to claim 1, wherein
the anti-fractalkine antibody is subcutaneously administered such that the mean trough concentration thereof is 20 μg/mL or higher.

10. A pharmaceutical composition for the treatment of rheumatoid arthritis,
the pharmaceutical composition comprising an anti-fractalkine antibody and a pharmaceutically acceptable excipient, wherein
the anti-fractalkine antibody is an antibody wherein:
a heavy chain variable region thereof comprises the amino acid sequence represented by SEQ ID NO: 13 (QVQLVQSGAEVKKPGASVKVSCKASGYTFTNYYIHWVKQAPGQGLEWIGWIYPGDGS PKFNERFKGRTTLTADKSTNTAYMLLSSLRSEDTAVYFCATGPTDGDYFDYWGQGTTV TVSS);
a light chain variable region thereof comprises the amino acid sequence represented by SEQ ID NO: 14 (DIQMTQSPSSLSASVGDRVTITCRASGNIHNFLAWYQQKPGKAPKLLIYNEKTLADGVP SRFSGSGSGTDYTLTISSLQPEDFATYFCQQFWSTPYTFGGGTKVEIK);
the antibody comprises a constant region of human IgG2 isotype; and
a Fc region of the constant region of human IgG2 isotype contains V234A and G237A mutations, and
the pharmaceutical composition is used such that at least 100 mg per dose of the anti-fractalkine antibody is subcutaneously administered to a human.

11. The pharmaceutical composition according to claim 10, wherein
the pharmaceutical composition is used such that 100 mg to 400 mg per dose of the anti-fractalkine antibody is subcutaneously administered to a human.

12. The pharmaceutical composition according to claim 10, wherein
the pharmaceutical composition is used such that 100 mg, 200 mg, or 400 mg per dose of the anti-fractalkine antibody is subcutaneously administered to a human.

13. The pharmaceutical composition according to claim 10, wherein
when 100 mg of the anti-fractalkine antibody is subcutaneously administered by a single administration to a human,
mean Cₘₐₓ is a value that falls within the numerical range of 80% to 125% of 10 μg/mL, mean AUC_{(0-336 h)} is a value that falls within the numerical range of 80% to 125% of 2.4×10³ μg·h/mL, or
mean AUC₍₀₋ₜ₎ is a value that falls within the numerical range of 80% to 125% of 3.0×10³ μg·h/mL.

14. The pharmaceutical composition according to claim 10, wherein
when 200 mg of the anti-fractalkine antibody is subcutaneously administered by a single administration to a human,
mean Cₘₐₓ is a value that falls within the numerical range of 80% to 125% of 27 μg/mL,
mean AUC_{(0-336 h)} is a value that falls within the numerical range of 80% to 125% of 7.4×10³ μg·h/mL, or
mean AUC₍₀₋ₜ₎ is a value that falls within the numerical range of 80% to 125% of 1.2×10⁴ μg·h/mL.

15. The pharmaceutical composition according to claim 10, wherein
when 400 mg of the anti-fractalkine antibody is subcutaneously administered by a single administration to a human,
mean Cₘₐₓ is a value that falls within the numerical range of 80% to 125% of 43 μg/mL,
mean AUC_{(0-336 h)} is a value that falls within the numerical range of 80% to 125% of 1.2×10⁴ μg·h/mL, or
mean AUC₍₀₋ₜ₎ is a value that falls within the numerical range of 80% to 125% of 2.7×10⁴ μg·h/mL.

16. The pharmaceutical composition according to claim 10, wherein
the pharmaceutical composition is subcutaneously administered by multiple administrations at dosing intervals of once a week to once every two weeks.

17. The pharmaceutical composition according to claim 10, wherein
the pharmaceutical composition is subcutaneously administered such that the mean trough concentration of the anti-fractalkine antibody is 10 μg/mL or higher.

18. The pharmaceutical composition according to claim 10, wherein
the pharmaceutical composition is subcutaneously administered such that the mean trough concentration of the anti-fractalkine antibody is 20 μg/mL or higher.


SEQUENCE LISTING (26 sequences)

SEQ ID NO: 1 - protein, 119 aa, Artificial Sequence
  Synthetic polypeptide
QVQLVQSGAEVKKPGASVKVSCKASGYTFTNYYIHWVKQAPGQGLEWIGWIYPGDGSPKFNERFKGRTTLTADKSTNTAYMLLSSLRSEDTAVYFCATGPTDGDYFDYWGQGTTVTVSS

SEQ ID NO: 2 - protein, 107 aa, Artificial Sequence
  Synthetic polypeptide
DIQMTQSPSSLSASVGDRVTITCRASGNIHNFLAWYQQKPGKAPKLLIYNEKTLADGVPSRFSGSGSGTDYTLTISSLQPEDFATYFCQQFWSTPYTFGGGTKVEIK

SEQ ID NO: 3 - protein, 19 aa, Artificial Sequence
  Synthetic polypeptide
MEWSWVFLFFLSVTTGVHS

SEQ ID NO: 4 - protein, 326 aa, Artificial Sequence
  Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 464 aa, Artificial Sequence
  Synthetic polypeptide
MEWSWVFLFFLSVTTGVHSQVQLVQSGAEVKKPGASVKVSCKASGYTFTNYYIHWVKQAPGQGLEWIGWIYPGDGSPKFNERFKGRTTLTADKSTNTAYMLLSSLRSEDTAVYFCATGPTDGDYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 20 aa, Artificial Sequence
  Synthetic polypeptide
MSVPTQVLGLLLLWLTDARC

SEQ ID NO: 7 - protein, 107 aa, Artificial Sequence [sequence omitted]

SEQ ID NO: 8 - protein, 234 aa, Artificial Sequence
  Synthetic polypeptide
MSVPTQVLGLLLLWLTDARCDIQMTQSPSSLSASVGDRVTITCRASGNIHNFLAWYQQKPGKAPKLLIYNEKTLADGVPSRFSGSGSGTDYTLTISSLQPEDFATYFCQQFWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1417 bp, Artificial Sequence
  Synthetic oligonucleotide
aagcttgccgccaccatggaatggtcctgggtgttcctgttcttcctgtccgtgaccaccggcgtgcactcccaggtgcagctggtgcagtctggcgccgaagtgaagaaacctggcgcctccgtgaaggtgtcctgcaaggcctccggctacaccttcaccaactactacatccactgggtgaaacaggccccaggacagggcctggaatggatcggctggatctaccccggcgacggctcccccaagttcaacgagcggttcaagggccggaccaccctgaccgccgacaagtccaccaacaccgcctacatgctgctgtcctccctgcggagcgaggataccgccgtgtacttctgcgccaccggccctaccgacggcgactacttcgactactggggccagggcaccaccgtgaccgtgtcctctgcctccaccaagggcccctccgtgttccctctggccccttgctcccggtccacctctgagtctaccgccgctctgggctgcctggtcaaagactacttccccgagcctgtgacagtgtcctggaactctggcgccctgacctctggagtgcataccttccctgccgtgctgcagtcatccggcctgtactccctgtcctccgtggtgacagtgccctcctccaacttcggcacccagacctacacctgtaacgtggaccacaagccctccaacaccaaggtggacaagaccgtggaacggaagtgctgcgtggaatgccccccctgtcctgcccctcctgccgccgctccttccgtgtttctgttccccccaaagcccaaggacaccctgatgatctcccggacccccgaagtgacctgcgtggtggtggacgtgtcccacgaggaccccgaggtgcagttcaattggtacgtggacggcgtggaagtgcacaacgccaagaccaagcccagagaggaacagttcaactccaccttccgggtggtgtccgtgctgaccgtggtgcaccaggactggctgaacggcaaagagtacaagtgcaaggtctccaacaagggcctgcctgcccccatcgaaaagaccatcagcaagaccaagggccagccccgcgagccccaggtgtacacactgccccccagccgggaagagatgaccaagaaccaggtgtccctgacctgtctggtgaaaggcttctacccctccgatatcgccgtggaatgggagtccaacggacagcccgagaacaactacaagaccaccccccccatgctggactccgacggctcattcttcctgtactccaagctgacagtggacaagtcccggtggcagcagggcaacgtgttctcctgcagcgtgatgcacgaggccctgcacaaccactacacccagaagtccctgagcctgagccccggcaagtgatgaattc

SEQ ID NO: 10 - DNA, 727 bp, Artificial Sequence
  Synthetic oligonucleotide
aagcttgccgccaccatgtccgtgcccacccaggtgctgggcctgctgctgctgtggctgaccgacgccagatgcgacatccagatgacccagtccccctccagcctgtccgcctctgtgggcgacagagtgaccatcacctgtcgggcctccggcaacatccacaactttctggcctggtatcagcagaagcccggcaaggcccccaagctgctgatctacaacgaaaagaccctggccgacggcgtgccctccagattctccggctctggctccggcaccgactacaccctgaccatctccagcctgcagcccgaggacttcgccacctacttttgccagcagttctggtccaccccctacaccttcggcggaggcaccaaggtggaaatcaagcggaccgtggccgctccctccgtgttcatcttcccaccctccgacgagcagctgaagtccggcaccgcctccgtggtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgctgatgaattc

SEQ ID NO: 11 - protein, 445 aa, Artificial Sequence
  Synthetic polypeptide
QVQLVQSGAEVKKPGASVKVSCKASGYTFTNYYIHWVKQAPGQGLEWIGWIYPGDGSPKFNERFKGRTTLTADKSTNTAYMLLSSLRSEDTAVYFCATGPTDGDYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Synthetic polypeptide
DIQMTQSPSSLSASVGDRVTITCRASGNIHNFLAWYQQKPGKAPKLLIYNEKTLADGVPSRFSGSGSGTDYTLTISSLQPEDFATYFCQQFWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, Artificial Sequence; identical to SEQ ID NO: 1
  Synthetic polypeptide

SEQ ID NO: 14 - protein, 107 aa, Artificial Sequence; identical to SEQ ID NO: 2
  Synthetic polypeptide

SEQ ID NO: 15 - protein, 5 aa, Artificial Sequence
  Synthetic polypeptide
NYYIH

SEQ ID NO: 16 - protein, 17 aa, Artificial Sequence
  Synthetic polypeptide
WIYPGDGSPKFNERFKG

SEQ ID NO: 17 - protein, 10 aa, Artificial Sequence
  Synthetic polypeptide
GPTDGDYFDY

SEQ ID NO: 18 - protein, 11 aa, Artificial Sequence
  Synthetic polypeptide
RASGNIHNFLA

SEQ ID NO: 19 - protein, 7 aa, Artificial Sequence
  Synthetic polypeptide
NEKTLAD

SEQ ID NO: 20 - protein, 9 aa, Artificial Sequence
  Synthetic polypeptide
QQFWSTPYT

SEQ ID NO: 21 - protein, 119 aa, Artificial Sequence
  Synthetic polypeptide
QVQLVQSGAEVKKPGASVKVSCKASGYTFTNYYIHWVKQAPGQGLEWIGWIYPGDGSPKFNERFKGRTTLTADKSTNTAYMLLSSLRSDDTAVYFCATGPTDGDYFDYWGQGTTVTVSS

SEQ ID NO: 22 - protein, 119 aa, Artificial Sequence
  Synthetic polypeptide
QVQLVQSGAEVKKPGASVKVSCKASGYTFTNYYIHWVRQAPGQGLEWIGWIYPGDGSPKFNERFKGRTTLTRDKSTNTAYMELSSLRSDDTAVYFCATGPTDGDYFDYWGQGTTVTVSS

SEQ ID NO: 23 - protein, 119 aa, Artificial Sequence
  Synthetic polypeptide
QVQLVQSGAEVKKPGASVKVSCKASGYTFTNYYIHWVRQAPGQGLEWIGWIYPGDGSPKFNERFKGRTTMTADTSTSTAYMELSSLRSEDTAVYFCARGPTDGDYFDYWGQGTTVTVSS

SEQ ID NO: 24 - protein, 119 aa, Artificial Sequence
  Synthetic polypeptide
QVQLVQSGAEVKKPGASVKVSCKASGYTFTNYYIHWVRQAPGQGLEWIGWIYPGDGSPKFNERFKGRTTLTADKSTSTAYMELSSLRSEDTAVYFCARGPTDGDYFDYWGQGTTVTVSS

SEQ ID NO: 25 - protein, 107 aa, Artificial Sequence
  Synthetic polypeptide
DIQMTQSPSSLSASVGDRVTITCRASGNIHNFLAWYQQKPGKAPKFLVYNEKTLADGVPSRFSGSGSGTQYTLTISSLQPEDFATYFCQQFWSTPYTFGGGTKVEIK

SEQ ID NO: 26 - protein, 107 aa, Artificial Sequence
  Synthetic polypeptide
DIQMTQSPSSLSASVGDRVTITCRASGNIHNFLAWYQQKPGKAPKLLIYNEKTLADGVPSRFSGSGSGTQYTLTISSLQPEDFATYFCQQFWSTPYTFGGGTKVEIK